(19) United States
(12) Patent Application Publication
(10) Pub. No.: US 2026/0009084 A1
(43) Pub. Date: Jan. 8, 2026

(54) BIOMARKER PANEL FOR DIAGNOSING CMS4 SUBTYPE OF COLORECTAL CANCER AND DIAGNOSTIC METHOD USING THE SAME

(71) Applicant: Korea Advanced Institute of Science and Technology, Daejeon (KR)

(72) Inventors: Pilnam KIM, Daejeon (KR); Hyun Jin LEE, Daejeon (KR)

(21) Appl. No.: 19/277,621

(22) Filed: Jul. 23, 2025

Related U.S. Application Data
(63) Continuation-in-part of application No. PCT/KR2024/001871, filed on Feb. 8, 2024.

(30) Foreign Application Priority Data
Feb. 10, 2023 (KR) 10-2023-0017914

(51) Int. Cl. C12Q 1/6886; G01N 33/574

(52) U.S. Cl. CPC C12Q 1/6886; G01N 33/57419; C12Q 2600/112; C12Q 2600/136; C12Q 2600/158; G01N 2500/00

(57) ABSTRACT

The present invention relates to biomarker panel for diagnosing CMS4 Subtype of colorectal cancer and diagnostic method using the same. Even among consensus molecular subtypes (CMSs) of colon cancer, CMS4 subtype is a group that exhibits significant changes in the expression of EMT-related genes and genes related to TGF-B signaling, angiogenesis, the activity of the complement-mediated inflammatory system, and stromal invasion, and is characterized by being the most incurable and poorly prognostic. The present invention is remarkably effective for accurately diagnosing the most difficult-to-treat and poorly prognostic types of colon cancer, and thus is expected to be widely used in the fields of medicine and health.

### TECHNICAL FIELD

The present disclosure relates to biomarker panel for diagnosing CMS4 Subtype of colorectal cancer and diagnostic method using the same.

### BACKGROUND ART

Colorectal cancer is a malignant tumor that occurs in the colon, and according to a report by the International Agency for Research on Cancer (IARC) under the World Health Organization (WHO), South Korea has the highest incidence rate in the world, with 45 cases per 100,000 people. According to cancer registration statistics published by the Korea Central Cancer Registry, a total of 217,057 cancer cases occurred in South Korea in 2014, of which colorectal cancer accounted for 26,978 cases, ranking third with 12.4% of the total. The mortality rate due to colorectal cancer is also high, ranking fourth among all cancer-related deaths. The incidence of colorectal cancer is increasing further due to longer life expectancy and the westernization of dietary habits. Therefore, highly accurate technologies for early detection of colorectal cancer are urgently needed to improve patient survival rates and quality of life.

Meanwhile, cancer classification is very important not only for accurate diagnosis but also because it allows for some prediction of the biological characteristics of each cancer. Although colorectal cancer shows relatively uniform clinical and morphological characteristics compared to other types of cancer, its biological features and progression are highly diverse, making precise classification essential for prediction. Recently, the World Health Organization (WHO) integrated previously sporadic data on molecular classification of colorectal cancer and categorized the disease into four subtypes, known as consensus molecular subtypes (CMS). Among them, subtype 4 (CMS4) is characterized by significant changes in the expression of genes related to epithelial-mesenchymal transition (EMT), TGF-β signaling, angiogenesis, activation of the complement-mediated inflammatory system, and stromal infiltration. This group is the most refractory and has the poorest prognosis.

Accordingly, the present invention was devised to solve the above problems, and provides a biomarker and a diagnostic method for effectively diagnosing colorectal cancer, particularly the CMS4 subtype. The present invention is expected to be widely used in the medical and healthcare fields, as it has a remarkable effect in diagnosing the most difficult-to-treat and prognostically unfavorable type of colorectal cancer with high accuracy.

### DISCLOSURE

### Technical Problem

One object of the present invention is to provide a composition or kit for diagnosing colorectal cancer.

Another object of the present invention is to provide a method for diagnosing or treating colorectal cancer.

Still another object of the present invention is to provide a method for screening a therapeutic agent for colorectal cancer.

However, objects to be achieved by the present disclosure are not limited to the objects mentioned above, and other objects not mentioned above may be clearly understood by those skilled in the art from the following description.

### Technical Solution

Hereinafter, various embodiments described herein will be described with reference to figures. In the following description, numerous specific details are set forth, such as specific configurations, compositions, and processes, etc., in order to provide a thorough understanding of the present disclosure. However, certain embodiments may be practiced without one or more of these specific details, or in combination with other known methods and configurations. In other instances, known processes and preparation techniques have not been described in particular detail in order to not unnecessarily obscure the present disclosure. Reference throughout this specification to “one embodiment” or “an embodiment” means that a particular feature, configuration, composition, or characteristic described in connection with the embodiment is included in at least one embodiment of the present disclosure. Thus, the appearances of the phrase “in one embodiment” or “an embodiment” in various places throughout this specification are not necessarily referring to the same embodiment of the present disclosure. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present disclosure pertains.

In this specification, the term “cancer” refers to uncontrolled cellular growth, characterized by the formation of a mass of abnormal cells, known as a tumor, which can invade surrounding tissues and, in severe cases, metastasize to other organs in the body. Scientifically, it is also referred to as a neoplasm. Despite treatment with surgery, radiation, and chemotherapy, cancer often cannot be fundamentally cured, causing significant suffering to patients and ultimately leading to death. Cancer is considered a chronic, intractable disease. Its causes are diverse and can be classified into intrinsic and extrinsic factors. Although the exact mechanisms by which normal cells transform into cancer cells have not been fully elucidated, it is known that many cancers are influenced by external factors such as environmental conditions. Intrinsic factors include genetic predisposition and immunological aspects, while extrinsic factors include chemical substances, radiation, and viruses. Genes involved in cancer development include oncogenes and tumor suppressor genes. Cancer arises when the balance between these genes is disrupted by intrinsic or extrinsic factors.

In this specification, the term “colorectal cancer” encompasses malignant tumors originating from the mucosa of the colon and rectum. It may present with polypoid, ulcerative, or infiltrative characteristics. Histologically, more than 90% of colorectal cancers are adenocarcinomas originating from the epithelial cells of the colonic mucosa. Less commonly, neuroendocrine carcinomas and squamous cell carcinomas may also occur. Adenocarcinomas are graded histologically based on the extent of gland formation: well-differentiated adenocarcinomas form glands in more than 95% of the tumor, moderately differentiated ones show glandular structures in 50-95%, and poorly differentiated tumors form glands in less than 50% of the tumor. Most colorectal adenocarcinomas are moderately differentiated, while well-differentiated types account for about 10% and poorly differentiated types about 20%. Recently, the World Health Organization (WHO) integrated previously sporadic molecular classification data of colorectal cancer and categorized the disease into four consensus molecular subtypes (CMS), the details of which are shown in Table 1 below.

TABLE 1

| Subtype | CMS1 | CMS2 | CMS3 | CMS4 |
|---|---|---|---|---|
| Dominant feature | MSI immune | Canonical | Metabolic | Mesenchymal |
| Prevalence | 14% | 37% | 13% | 23% |
| Genome instability | MSI high | SCNA high | Mixed MSI | SCNA high |
| | CIMP high | | CIMP low | |
| | Hypermutation | | SCNA low | |
| Mutation | BRAF | | KRAS | |
| Pathway and | Immune | WNT and | Metabolic | Stromal |
| microenvironment | activation | MYC | dysregulation | invasion |
| | | activation | | TGF-β |
| | | | | activation |
| | | | | angiogenesis |
| Prognostic | Worse SAR | | | Worse RFS and |
| | | | | OS |

MSI, microsatellite instable;
CIMP, CpG island methylator phenotype;
SAR, survival after relapse;
SCNA, somatic copy number alteration;
WNT, wingless-type MMTV integration site;
MYC, v-myc avian myelocytomatosis viral oncogene;
TGF-β, transforming growth factor β;
RFS, relapse-free survival;
OS, overall survival.

In this specification, the term “diagnosis” refers to the identification of the presence or characteristics of a pathological condition. For the purposes of the present invention, the diagnosis refers to determining the onset or the likelihood of onset of colorectal cancer, particularly CMS4 subtype colorectal cancer, thereby enabling early prediction of the occurrence of colorectal cancer, especially the CMS4 subtype.

In this specification, the genes described as biomarkers are human (Homo sapiens)-derived genes, and information on these genes can be readily retrieved from public databases that are well known to those skilled in the art, such as the National Center for Biotechnology Information (NCBI).

According to one embodiment of the present invention, the invention relates to biomarkers for the diagnosis of colorectal cancer, particularly the CMS4 subtype of colorectal cancer.

The biomarker may be at least one genes, or a protein encoded thereby, selected from the group consisting of COL14A1 (Collagen Type XIV Alpha 1 Chain), DPT (Dermatopontin), MFAP5 (Microfibril Associated Protein 5), MATN2 (Matrilin-2), SRPX (Sushi Repeat Containing Protein X-Linked), MFAP4 (Microfibril Associated Protein 4), MGP (Matrix Gla Protein), TNXB (tenascin XB protein), EDIL3 (EGF Like Repeats And Discoidin Domains 3), LTBP4 (latent transforming growth factor beta binding protein 4), SPARCL1 (SPARC Like 1), OGN (Osteoglycin), HAPLN1 (Hyaluronan And Proteoglycan Link Protein 1), DCN (Decorin), ADAMDEC1 (ADAM like decysin 1), A2M (Alpha-2-Macroglobulin), CTSC (Cathepsin C), CST3 (cystatin c), CXCL12 (C-X-C motif chemokine 12), and S100A4 (S100 Calcium Binding Protein A4). The one or more genes or proteins selected from the above may exhibit decreased expression levels in colorectal cancer, particularly the CMS4 subtype, compared to normal controls.

Alternatively, the biomarker may be at least one genes, or a protein encoded thereby, selected from the group consisting of COL12A1 (Collagen type XII α1 chain), COL11A1 (Collagen Type XI Alpha 1 Chain), CTHRC1 (Collagen Triple Helix Repeat Containing 1), FN1 (Fibronectin 1), TNC (Tenascin C), SPARC (Secreted Protein Acidic And Cysteine Rich), THBS2 (Thrombospondin 2), TIMP1 (TIMP Metallopeptidase Inhibitor 1), MMP14 (Matrix Metallopeptidase 14), PLOD2 (Procollagen-Lysine, 2-Oxoglutarate 5-Dioxygenase 2), SERPINH1 (Serpin peptidase inhibitor clade H, member 1), LOXL2 (Lysyl Oxidase Like 2), MMP11 (Matrix Metallopeptidase 11), MMP1 (Matrix Metallopeptidase 1), CTSB (Cathepsin B), MMP3 (Matrix Metallopeptidase 3), LGALS1 (Galectin 1), and SFRP4 (Secreted Frizzled Related Protein 4). The one or more genes or proteins selected from the above may exhibit increased expression levels in colorectal cancer, particularly the CMS4 subtype, compared to normal controls.

The biomarker for diagnosing colorectal cancer, particularly the CMS4 subtype of colorectal cancer, according to the present invention, may comprise at least one genes, or a protein encoded thereby, selected from the group consisting of COL14A1 (Collagen Type XIV Alpha 1 Chain), DPT (Dermatopontin), MFAP5 (Microfibril Associated Protein 5), MATN2 (Matrilin-2), SRPX (Sushi Repeat Containing Protein X-Linked), MFAP4 (Microfibril Associated Protein 4), MGP (Matrix Gla Protein), TNXB (tenascin XB protein), EDIL3 (EGF Like Repeats And Discoidin Domains 3), LTBP4 (latent transforming growth factor beta binding protein 4), SPARCL1 (SPARC Like 1), OGN (Osteoglycin), HAPLN1 (Hyaluronan And Proteoglycan Link Protein 1), DCN (Decorin), ADAMDEC1 (ADAM like decysin 1), A2M (Alpha-2-Macroglobulin), CTSC (Cathepsin C), CST3 (cystatin c), CXCL12 (C-X-C motif chemokine 12), and S100A4 (S100 Calcium Binding Protein A4), and may further comprise at least one genes, or a protein encoded thereby, selected from the group consisting of COL12A1 (Collagen type XII α1 chain), COL11A1 (Collagen Type XI Alpha 1 Chain), CTHRC1 (Collagen Triple Helix Repeat Containing 1), FN1 (Fibronectin 1), TNC (Tenascin C), SPARC (Secreted Protein Acidic And Cysteine Rich), THBS2 (Thrombospondin 2), TIMP1 (TIMP Metallopeptidase Inhibitor 1), MMP14 (Matrix Metallopeptidase 14), PLOD2 (Procollagen-Lysine, 2-Oxoglutarate 5-Dioxygenase 2), SERPINH1 (Serpin peptidase inhibitor clade H, member 1), LOXL2 (Lysyl Oxidase Like 2), MMP11 (Matrix Metallopeptidase 11), MMP1 (Matrix Metallopeptidase 1), CTSB (Cathepsin B), MMP3 (Matrix Metallopeptidase 3), LGALS1 (Galectin 1), and SFRP4 (Secreted Frizzled Related Protein 4). In such cases, the diagnostic accuracy for colorectal cancer, particularly the CMS4 subtype of colorectal cancer, may be improved.

According to another embodiment of the present invention, the invention relates to a composition for diagnosing colorectal cancer, particularly the CMS4 subtype of colorectal cancer.

The diagnostic composition may comprise an agent for measuring the expression level of at least one gene, or a protein encoded thereby, selected from the group consisting of COL14A1 (Collagen Type XIV Alpha 1 Chain), DPT (Dermatopontin), MFAP5 (Microfibril Associated Protein 5), MATN2 (Matrilin-2), SRPX (Sushi Repeat Containing Protein X-Linked), MFAP4 (Microfibril Associated Protein 4), MGP (Matrix Gla Protein), TNXB (tenascin XB protein), EDIL3 (EGF Like Repeats And Discoidin Domains 3), LTBP4 (latent transforming growth factor beta binding protein 4), SPARCL1 (SPARC Like 1), OGN (Osteoglycin), HAPLN1 (Hyaluronan And Proteoglycan Link Protein 1), DCN (Decorin), ADAMDEC1 (ADAM like decysin 1), A2M (Alpha-2-Macroglobulin), CTSC (Cathepsin C), CST3 (cystatin c), CXCL12 (C-X-C motif chemokine 12), and S100A4 (S100 Calcium Binding Protein A4).

Alternatively, the diagnostic composition may further comprise an agent for measuring the expression level of at least one genes, or a protein encoded thereby, selected from the group consisting of COL12A1 (Collagen type XII α1 chain), COL11A1 (Collagen Type XI Alpha 1 Chain), CTHRC1 (Collagen Triple Helix Repeat Containing 1), FN1 (Fibronectin 1), TNC (Tenascin C), SPARC (Secreted Protein Acidic And Cysteine Rich), THBS2 (Thrombospondin 2), TIMP1 (TIMP Metallopeptidase Inhibitor 1), MMP14 (Matrix Metallopeptidase 14), PLOD2 (Procollagen-Lysine, 2-Oxoglutarate 5-Dioxygenase 2), SERPINH1 (Serpin peptidase inhibitor clade H, member 1), LOXL2 (Lysyl Oxidase Like 2), MMP11 (Matrix Metallopeptidase 11), MMP1 (Matrix Metallopeptidase 1), CTSB (Cathepsin B), MMP3 (Matrix Metallopeptidase 3), LGALS1 (Galectin 1), and SFRP4 (Secreted Frizzled Related Protein 4).

In the present invention, the agent for measuring the expression level of the above-mentioned protein is not particularly limited but may include, for example, at least one selected from the group consisting of antibodies, oligopeptides, ligands, peptide nucleic acids (PNAs), and aptamers that specifically bind to the protein.

As used herein, the term “antibody” refers to a substance that specifically binds to an antigen to elicit an antigen-antibody reaction. For the purposes of the present invention, the antibody refers to one that specifically binds to the biomarker protein. The antibody of the present invention includes polyclonal antibodies, monoclonal antibodies, and recombinant antibodies. Such antibodies can be easily prepared using techniques well known in the art. For example, polyclonal antibodies can be produced using a method well known in the art, which comprises injecting an antigen derived from the biomarker protein into an animal, collecting blood from the animal, and obtaining serum containing the antibody. Such polyclonal antibodies may be prepared from any suitable animal such as goat, rabbit, sheep, monkey, horse, pig, cow, or dog. Furthermore, monoclonal antibodies can be produced by hybridoma techniques or phage display library techniques, both of which are widely known in the art. Antibodies prepared by such methods may be isolated and purified using techniques such as gel electrophoresis, dialysis, salt precipitation, ion exchange chromatography, or affinity chromatography. The antibodies of the present invention may include not only the complete form comprising two full-length light chains and two full-length heavy chains, but also functional fragments of the antibody molecule. A functional fragment of the antibody refers to a fragment that retains at least antigen-binding function, such as Fab, F(ab′), F(ab′)₂, and Fv.

The term “PNA (Peptide Nucleic Acid)” as used in the present invention encompasses artificially synthesized polymers similar to DNA or RNA. Unlike DNA, which has a phosphate-ribose backbone, PNA has a backbone composed of repeating N-(2-aminoethyl)-glycine units linked by peptide bonds. This structure results in significantly enhanced binding affinity and stability to DNA or RNA, making PNAs useful in molecular biology, diagnostic analysis, and antisense therapy.

The term “aptamer” as used in the present invention refers to an oligonucleotide or peptide molecule. Aptamers can be prepared by various methods known to those skilled in the art.

In the present invention, the agent for measuring the expression level of the gene encoding the above-mentioned protein may include at least one selected from the group consisting of primers, probes, and antisense nucleotides that specifically bind to the gene.

The term “primer” as used in the present invention refers to a short nucleic acid fragment that recognizes a target gene sequence and includes both forward and reverse primers. Preferably, the primers are capable of providing analytical results with high specificity and sensitivity. High specificity can be conferred when the nucleic acid sequence of the primer does not match non-target sequences present in the sample and amplifies only the target gene sequence containing a complementary primer binding site, thereby avoiding non-specific amplification.

In the present invention, the term “probe” refers to a substance that can specifically bind to a target material to be detected within a sample, and through such binding, specifically confirm the presence of the target material within the sample. The type of probe is not limited and may be any material commonly used in the relevant art, preferably including peptide nucleic acid (PNA), locked nucleic acid (LNA), peptides, polypeptides, proteins, RNA, or DNA, with PNA being most preferred. More specifically, the probe may be a biological material derived from or similar to those derived from living organisms, or produced ex vivo. Examples include enzymes, proteins, antibodies, microorganisms, plant and animal cells and tissues, nerve cells, DNA, and RNA. DNA may include cDNA, genomic DNA, and oligonucleotides; RNA may include genomic RNA, mRNA, and oligonucleotides; and proteins may include antibodies, antigens, enzymes, peptides, and the like.

In the present invention, “LNA (Locked Nucleic Acids)” refers to nucleic acid analogues containing a 2′-O, 4′-C methylene bridge. LNA nucleosides include the common nucleobases of DNA and RNA and can form base pairs according to Watson-Crick base pairing rules. However, due to the ‘locking’ effect caused by the methylene bridge, LNA does not adopt the ideal conformation typical of Watson-Crick base pairing. When LNA is incorporated into DNA or RNA oligonucleotides, it pairs more rapidly with complementary nucleotide strands, thereby increasing the stability of the double helix.

In the present invention, the term “antisense” refers to antisense oligomers that hybridize to a target sequence within RNA by Watson-Crick base pairing, typically allowing the formation of RNA: oligomer heteroduplexes within the target sequence. These oligomers have nucleotide bases and backbones between subunits and may exhibit exact or approximate sequence complementarity to the target sequence.

Since the information on the biomarker proteins according to the present invention, or the genes encoding them, is known, those skilled in the art can readily design primers, probes, or antisense nucleotides that specifically bind to the genes encoding the proteins based on this information.

In the composition for diagnosing of the present invention, the expression level of at least one gene, or a protein encoded thereby, for colorectal cancer, particularly CMS4 subtype colorectal cancer, may be measured in decellularized tissue, and more specifically, may be measured in the decellularized extracellular matrix.

In the composition for diagnosing of the present invention, when the expression level of at least one genes, or a protein encoded thereby, selected from the group consisting of COL14A1 (Collagen Type XIV Alpha 1 Chain), DPT (Dermatopontin), MFAP5 (Microfibril Associated Protein 5), MATN2 (Matrilin-2), SRPX (Sushi Repeat Containing Protein X-Linked), MFAP4 (Microfibril Associated Protein 4), MGP (Matrix Gla Protein), TNXB (tenascin XB protein), EDIL3 (EGF Like Repeats And Discoidin Domains 3), LTBP4 (latent transforming growth factor beta binding protein 4), SPARCL1 (SPARC Like 1), OGN (Osteoglycin), HAPLN1 (Hyaluronan And Proteoglycan Link Protein 1), DCN (Decorin), ADAMDEC1 (ADAM like decysin 1), A2M (Alpha-2-Macroglobulin), CTSC (Cathepsin C), CST3 (cystatin c), CXCL12 (C-X-C motif chemokine 12), and S100A4 (S100 Calcium Binding Protein A4) is decreased compared to a normal control group, it can be diagnosed that there is a high likelihood of colorectal cancer, particularly CMS4 subtype colorectal cancer.

In the composition for diagnosing of the present invention, when the expression level of at least one genes, or a protein encoded thereby, selected from the group consisting of COL12A1 (Collagen type XII α1 chain), COL11A1 (Collagen Type XI Alpha 1 Chain), CTHRC1 (Collagen Triple Helix Repeat Containing 1), FN1 (Fibronectin 1), TNC (Tenascin C), SPARC (Secreted Protein Acidic And Cysteine Rich), THBS2 (Thrombospondin 2), TIMP1 (TIMP Metallopeptidase Inhibitor 1), MMP14 (Matrix Metallopeptidase 14), PLOD2 (Procollagen-Lysine, 2-Oxoglutarate 5-Dioxygenase 2), SERPINH1 (Serpin peptidase inhibitor clade H, member 1), LOXL2 (Lysyl Oxidase Like 2), MMP11 (Matrix Metallopeptidase 11), MMP1 (Matrix Metallopeptidase 1), CTSB (Cathepsin B), MMP3 (Matrix Metallopeptidase 3), LGALS1 (Galectin 1), and SFRP4 (Secreted Frizzled Related Protein 4) is increased compared to a normal control group, it can be diagnosed that there is a high likelihood of colorectal cancer, particularly CMS4 subtype colorectal cancer.

In the composition for diagnosing colorectal cancer, particularly the CMS4 subtype of colorectal cancer, according to the present invention, may comprise an agent for measuring the expression level of at least one gene, or a protein encoded thereby, selected from the group consisting of COL14A1 (Collagen Type XIV Alpha 1 Chain), DPT (Dermatopontin), MFAP5 (Microfibril Associated Protein 5), MATN2 (Matrilin-2), SRPX (Sushi Repeat Containing Protein X-Linked), MFAP4 (Microfibril Associated Protein 4), MGP (Matrix Gla Protein), TNXB (tenascin XB protein), EDIL3 (EGF Like Repeats And Discoidin Domains 3), LTBP4 (latent transforming growth factor beta binding protein 4), SPARCL1 (SPARC Like 1), OGN (Osteoglycin), HAPLN1 (Hyaluronan And Proteoglycan Link Protein 1), DCN (Decorin), ADAMDEC1 (ADAM like decysin 1), A2M (Alpha-2-Macroglobulin), CTSC (Cathepsin C), CST3 (cystatin c), CXCL12 (C-X-C motif chemokine 12), and S100A4 (S100 Calcium Binding Protein A4), and may further comprise an agent for measuring the expression level of at least one genes, or a protein encoded thereby, selected from the group consisting of COL12A1 (Collagen type XII α1 chain), COL11A1 (Collagen Type XI Alpha 1 Chain), CTHRC1 (Collagen Triple Helix Repeat Containing 1), FN1 (Fibronectin 1), TNC (Tenascin C), SPARC (Secreted Protein Acidic And Cysteine Rich), THBS2 (Thrombospondin 2), TIMP1 (TIMP Metallopeptidase Inhibitor 1), MMP14 (Matrix Metallopeptidase 14), PLOD2 (Procollagen-Lysine, 2-Oxoglutarate 5-Dioxygenase 2), SERPINH1 (Serpin peptidase inhibitor clade H, member 1), LOXL2 (Lysyl Oxidase Like 2), MMP11 (Matrix Metallopeptidase 11), MMP1 (Matrix Metallopeptidase 1), CTSB (Cathepsin B), MMP3 (Matrix Metallopeptidase 3), LGALS1 (Galectin 1), and SFRP4 (Secreted Frizzled Related Protein 4). In such cases, the diagnostic accuracy for colorectal cancer, particularly the CMS4 subtype of colorectal cancer, may be improved.

According to another embodiment of the present invention, the invention relates to a colorectal cancer diagnostic kit comprising a diagnostic composition for colorectal cancer, particularly CMS4 subtype colorectal cancer.

The kit of the present invention includes the diagnostic composition for colorectal cancer, particularly CMS4 subtype colorectal cancer, as described above. The limitations on each component constituting the diagnostic composition of the present invention overlap with those described for the diagnostic composition for colorectal cancer, particularly CMS4 subtype colorectal cancer, and therefore are omitted herein to avoid excessive complexity in this specification.

In the present invention, the kit may be an RT-PCR kit, DNA chip kit, ELISA kit, protein chip kit, rapid kit, or MRM (Multiple Reaction Monitoring) kit, but is not limited thereto.

The diagnostic kit of the present invention may further include one or more other constituent compositions, solutions, or devices suitable for the analytical method. For example, the diagnostic kit of the present invention may further include essential components required to perform a reverse transcription polymerase reaction. The reverse transcription polymerase reaction kit includes primer pairs specific for genes encoding marker proteins. The primers are nucleotides having sequences specific to the nucleic acid sequences of the genes, with lengths of about 7 bp to 50 bp, preferably about 10 bp to 30 bp. It may also include primers specific to the nucleic acid sequences of control genes. Additionally, the reverse transcription polymerase reaction kit may include test tubes or other suitable containers, reaction buffers (with varying pH and magnesium concentrations), deoxynucleotides (dNTPs), enzymes such as Taq polymerase and reverse transcriptase, DNase, RNase inhibitors, DEPC-water, sterile water, and the like. Moreover, the diagnostic kit of the present invention may include essential components necessary to perform DNA chip analysis. The DNA chip kit may include substrates to which cDNA or oligonucleotides corresponding to the gene or its fragment are attached, reagents, formulations, enzymes for preparing fluorescently labeled probes, and the like. The substrate may also include cDNA or oligonucleotides corresponding to control genes or their fragments. Furthermore, the diagnostic kit of the present invention may include essential components necessary to perform ELISA. The ELISA kit includes antibodies specific to the proteins. The antibodies have high specificity and affinity for the marker proteins and exhibit minimal cross-reactivity with other proteins; they may be monoclonal antibodies, polyclonal antibodies, or recombinant antibodies. The ELISA kit may also include antibodies specific to control proteins. Other components of the ELISA kit may include reagents capable of detecting bound antibodies, such as labeled secondary antibodies, chromophores, enzymes (e.g., conjugated to antibodies), substrates for the enzymes, or other materials capable of binding antibodies.

According to another embodiment of the present invention, the invention relates to a method for diagnosing colorectal cancer, particularly CMS4 subtype colorectal cancer, comprising a step of measuring the expression level of at least one gene, or a protein encoded thereby, selected from the biomarkers described herein, in a biological sample isolated from a subject of interest.

In the present invention, the term “subject of interest” refers to a subject whose colorectal cancer status is uncertain or whose colorectal cancer has been diagnosed but whose CMS subtype is unclear.

In the present invention, the term “biological sample” refers to any material, biological fluid, tissue, or cells obtained from or derived from the subject, preferably colorectal tissue, which is desirable for improving the accuracy of colorectal cancer diagnosis.

The present invention may include the step of measuring the expression level of the listed biomarker proteins or the genes encoding them in the isolated biological sample as described above. The step of measuring the expression level of the selected proteins or genes, or the reagents capable of measuring the expression level of the selected proteins or genes, overlap with those described in the diagnostic composition for colorectal cancer, particularly CMS4 subtype colorectal cancer, and thus are omitted herein to avoid excessive complexity in this specification.

According to another embodiment of the present invention, the invention relates to a method for screening candidate therapeutic agents for colorectal cancer, particularly CMS4 subtype colorectal cancer.

Specifically, the method may include: (a) a step of treating a biological sample isolated from a target subject with a candidate therapeutic agent for colorectal cancer; and (b) a step of measuring the expression level of at least one gene, or a protein encoded thereby, selected from the group consisting of COL14A1 (Collagen Type XIV Alpha 1 Chain), DPT (Dermatopontin), MFAP5 (Microfibril Associated Protein 5), MATN2 (Matrilin-2), SRPX (Sushi Repeat Containing Protein X-Linked), MFAP4 (Microfibril Associated Protein 4), MGP (Matrix Gla Protein), TNXB (tenascin XB protein), EDIL3 (EGF Like Repeats And Discoidin Domains 3), LTBP4 (latent transforming growth factor beta binding protein 4), SPARCL1 (SPARC Like 1), OGN (Osteoglycin), HAPLN1 (Hyaluronan And Proteoglycan Link Protein 1), DCN (Decorin), ADAMDEC1 (ADAM like decysin 1), A2M (Alpha-2-Macroglobulin), CTSC (Cathepsin C), CST3 (cystatin c), CXCL12 (C-X-C motif chemokine 12), and S100A4 (S100 Calcium Binding Protein A4); and wherein if the expression level of the selected proteins or genes measured in step (b) is increased compared to before treatment with the candidate agent, the candidate agent may be determined to be a therapeutic agent for colorectal cancer.

The screening method may further include: (c) a step of measuring the expression level of at least one gene, or a protein encoded thereby, selected from the group consisting of COL12A1 (Collagen type XII α1 chain), COL11A1 (Collagen Type XI Alpha 1 Chain), CTHRC1 (Collagen Triple Helix Repeat Containing 1), FN1 (Fibronectin 1), TNC (Tenascin C), SPARC (Secreted Protein Acidic And Cysteine Rich), THBS2 (Thrombospondin 2), TIMP1 (TIMP Metallopeptidase Inhibitor 1), MMP14 (Matrix Metallopeptidase 14), PLOD2 (Procollagen-Lysine, 2-Oxoglutarate 5-Dioxygenase 2), SERPINH1 (Serpin peptidase inhibitor clade H, member 1), LOXL2 (Lysyl Oxidase Like 2), MMP11 (Matrix Metallopeptidase 11), MMP1 (Matrix Metallopeptidase 1), CTSB (Cathepsin B), MMP3 (Matrix Metallopeptidase 3), LGALS1 (Galectin 1), and SFRP4 (Secreted Frizzled Related Protein 4); and wherein if the expression level of the selected proteins or genes measured in step (c) is decreased compared to before treatment with the candidate agent, the candidate agent may be determined to be a therapeutic agent for colorectal cancer.

The reagents and methods for measuring expression levels used in the screening method of the present invention overlap with those described in the diagnostic method of the present invention and thus are omitted herein to avoid excessive complexity of the specification.

According to another embodiment of the present invention, the invention relates to a method for treating colorectal cancer, particularly the CMS4 subtype of colorectal cancer, comprising the step of measuring the expression level of at least one gene, or a protein encoded thereby, selected from biomarkers described herein or a protein encoded thereby in a biological sample isolated from a subject of interest.

Furthermore, the method of the present invention may further comprise the step of treating the subject of interest if, as described above, the subject is predicted or diagnosed to be at high risk of developing or progressing colorectal cancer, particularly the CMS4 subtype. The treatment may involve administering an appropriate therapeutic agent (e.g., a drug for the disease), performing surgery, or a combination thereof. The therapeutic agent may be a conventionally used drug or a candidate compound.

By using the method of the present invention, it is possible to diagnose the onset or risk of progression of colorectal cancer, particularly the CMS4 subtype, and to monitor disease progression, prognosis, or the therapeutic efficacy of a treatment.

Hereinafter, the present invention will be described in detail with reference to examples.

### Advantageous Effects

The present invention is expected to be widely used in the medical and healthcare fields, as it has a remarkable effect in diagnosing the most difficult-to-treat and prognostically unfavorable type of colorectal cancer with high accuracy.

### DESCRIPTION OF DRAWINGS

FIG. 1 is a schematic diagram of an overview of the characterization study of patient-derived ECM (pdECM). Patient-derived samples were decellularized to enrich extracellular matrix (ECM), and the proteomic profile of the pdECM was quantitatively analyzed using TMT (tandem mass tag) mass spectrometry. Single-cell and bulk RNA sequencing data were integrated to analyze the matrisome and heterogeneity of tissue-derived fibroblasts.

FIG. 2 shows the clinical data including consensus molecular subtype (CMS), sample type, tumor stage, and anatomical region of bulk tissue samples.

FIG. 3 shows the hematoxylin and eosin staining results of non-decellularized or decellularized patient-derived ECM. In FIG. 3, the scale bars represent 1 cm (white) or 100 μm (black).

FIG. 4 shows the quantification of DNA in non-decellularized or decellularized patient-derived ECM. In FIG. 4, ***: p<0.001.

FIG. 5 shows a qualitative comparison of matrisome proteins detected in previous studies and the present study.

FIG. 6 shows the relative percentage composition (RPC) of proteins detected in the reference sample with category-specific annotations of the matrisome. The number of proteins in each category is indicated in parentheses.

FIG. 7 shows the results of gene ontology analysis of the top 100 proteins with the highest intensity in category-specific RPCs. In FIG. 7, bar graphs represent the number of proteins, and dots indicate statistical significance for each category.

FIG. 8 shows the composition of core proteins across samples as represented by hierarchical clustering heatmaps and bar graphs of the matrisome-enriched proteomic profiles from patient-derived normal and tumor ECM. The hierarchical clustering demonstrates clear separation between normal and tumor groups and heterogeneity within the tumor group. RPCs of all annotated proteins between each sample and the average of normal/tumor states are shown in the bar graphs.

FIG. 9 shows a PCA plot of all samples. Replicates of tumor samples are plotted close to their original samples and are displayed in lighter colors.

FIG. 10 shows the protein distribution of the normal and tumor groups. Detected proteins were ranked by RPC, and the bar graph displays the 20 most abundant matrisome proteins in each group. The RPC of each protein in each sample is represented by dots on the bar graph.

FIG. 11 shows a volcano plot of differentially expressed proteins (DEPs) between patient-derived normal ECM and tumor ECM matrisomes. In FIG. 11, the red line indicates the threshold for DEP with log2(fold change)>0.5 and adjusted p<0.01. Twenty-eight tumor-enriched DEPs are shown on the right, and 110 normal-enriched DEPs are shown on the left.

FIG. 12 shows functional gene set enrichment analysis of the DEPs. In FIG. 12, the bar graph indicates the most annotated functions with statistical significance for normal-enriched and tumor-enriched DEPs.

FIG. 13 shows a heatmap of selected DEPs included in core matrix categories. Due to the heterogeneous matrisome composition in the tumor group, the expression patterns of tumor-enriched DEPs show inconsistent profiles across samples compared to normal-enriched DEPs.

FIG. 14 shows the cellular origin analysis of DEPs. The cellular origin of the DEPs was determined based on cell-type-specific expression patterns and the highest average expression level using single-cell sequencing data. In FIG. 14, the bar graph shows the cellular origin of DEPs along with the number and proportion of each cell type. Most DEPs were derived from fibroblasts.

FIG. 15 shows tSNE plots of single-cell sequencing results for normal and tumor colorectal tissues. Cells were clustered based on transcriptomic profiles, and tumor-and normal-associated clusters were grouped into metaclusters for each group. Differentially expressed matrisome genes were defined as tumor-associated markers (TAM) and normal-associated markers (NAM).

FIG. 16 shows protein and transcript expression of TAM and NAM. TAM and NAM were defined based on proteomic and transcriptomic expression data. In FIG. 16, the heatmap displays protein profiles of TAM and NAM, and the dot plot shows transcript profiles of TAM and NAM with their major expressing cell types.

FIG. 17 shows immunohistochemical images of COL12A1-, THBS2-, and HAPLN1-stained normal and tumor tissues. In FIG. 17, the scale bar represents 50 μm.

FIG. 18 shows normalized expression scores of TAM and NAM along with the consensus molecular subtype (CMS) of each tissue. In FIG. 18, the left panel shows TAM scores, and the right panel shows NAM scores. The black lines inside each box indicate the median score of each group. Statistically significant differences are denoted by lowercase letters.

FIG. 19 shows a GSEA plot of 38 matrisome markers. Using enrichment scores, 29 CMS4-enriched markers were identified.

FIG. 20 shows scatter plots indicating a positive correlation between the scores of the 29 CMS4-enriched markers and either EMT scores or TGF-β response scores.

FIG. 21 shows PFS (progression-free survival) from TCGA samples based on the expression patterns of the top 10 selected CMS4-specific matrisome markers, CMS4 probability, and 10-marker scores. Ten clinically significant markers with p-values <0.05 between the top 25% high-expression group and bottom 25% low-expression group were selected. CMS4 probability and 10-marker scores were calculated using the CMS4 classifier R package and ssGSEA.

FIG. 22 shows a heatmap indicating a positive correlation between the expression levels of the top 10 markers and CMS4 probability in TCGA samples.

FIG. 23 shows scatter plots indicating a positive correlation between the expression levels of the top 10 markers and CMS4 probability in TCGA samples.

### BEST MODE

The consensus molecular subtype (CMS), a classification based on transcriptional profiles, was recently developed to emphasize the importance of the ECM microenvironment in CRC. The CMS describes four CRC subtypes, among which the mesenchymal subtype or CMS4 group is characterized by extensive stromal infiltration (mostly activated fibroblasts) and ECM organization. Recent studies have demonstrated that CAFs in CRCs are composed of distinct fibroblast populations and significantly enriched in the CMS4 subtype compared with the other subtypes. Therefore, we sought to compare ECM features between the myofibroblast-enriched CMS4 subtype and other subtypes. We performed single-sample gene set enrichment analysis (ssGSEA) with The Cancer Genome Atlas (TCGA)-Colon Adenocarcinoma (COAD)/Rectal Adenocarcinoma (READ) expression data sets to calculate the expression patterns of TAM and NAM. The TAM ssGSEA scores were significantly higher in the stroma-enriched molecular subtype (CMS4) than in other cell types. The NAM scores were higher in normal tissues than in tumor tissues, and the scores varied according to tumor tissues. In particular, the levels of NAM were higher in the CMS4 subtype than in other subtypes, but the transcript levels of NAM were slightly lower in the CMS4 subtype than in normal tissues. Overall, fibroblasts from CMS4 showed increased transcript levels of most ECM genes, which is consistent with the molecular features of ECM organization and stromal invasion. Thus, the 10clinically significant CMS4-specific matrisome genes may be used to infer the fibroblast population in the TME and to discriminate between CMS4 and other subtypes.

### Mode for Invention

Hereinafter, the present disclosure will be described in detail with reference to the following examples. However, the following examples are merely illustrative of the present disclosure, and the content of the present disclosure is not limited by the following examples.

### EXAMPLE

### Methods

### Patient and Tissue Sample Collection

Tissue samples were obtained from patients diagnosed with colorectal cancer based on colonoscopic findings. In some patients, normal tissues were also collected in conjunction with the matched colorectal cancer tissues. The tissues harvested immediately after surgery were promptly preprocessed and stored frozen. The clinical characteristics of all patients and tissue samples were documented based on medical records and interviews.

### Tissue Decellularization Process

Collected tissues were decellularized using a detergent-based method. The following decellularizing detergent solution was used to remove the cellular components from tissues: 1% (v/v) Triton X-100 (T8787; Sigma-Aldrich, St. Louis, MO, USA) and 0.1% (v/v) ammonium hydroxide (221228; SigmaAldrich) in distilled water. Tissue samples were cut into small sections (3×3×3 mm) and treated with decellularizing solution for >2 h; the solution was replaced at 30-min intervals or when it became opaque. When the tissue became colorless, the resulting pdECM samples were washed with Dulbecco's phosphate buffered-saline (Welgene, Gyeongsan, Korea) for 2 days; the solution was replaced at 1-h intervals. Then, the tissue was washed with distilled water, 4 times for 10 min each, to remove residual Dulbecco's phosphate buffered-saline. Decellularization was performed on an orbital shaker at room temperature, using a speed of 70 rpm. Finally, pdECM samples were lyophilized for 1 day and stored at −20° C. until use. Hereinafter, the decellularized patient-derived tissues are referred to as pdECM (patient-derived extracellular matrix).

### Decellularized Tissue Characterization

For hematoxylin and eosin staining, native tissues and decellularized tissues were fixed in 4% paraformaldehyde (Biosesang, Seongnam, Korea) for 1 day and embedded in Paraplast (Leica Biosystems, Wetzlar, Germany); each sample was cut into 10-um-thick sections. The sectioned samples were stained with hematoxylin and eosin using the standard protocol with slight modification. The DNA content in pdECM samples was quantified using the DNA extraction kit (Bioneer, Daejeon, Korea) in accordance with the manufacturer's recommendations, and DNA concentrations were measured using a DS-11 Spectrophotometer (DeNovix, Wilmington, DE, USA).

### S-Trap Protein Digestion

The S-Trap™ mini (ProtiFi, Huntington, NY, USA) was used to perform protein digestion, in accordance with a slightly modified version of the manufacturer's instructions. Briefly, nearly 5 mg of decellularized colon tissues were mixed with 5% sodium dodecyl sulfate buffer and sonicated by VCX 130 (Sonics), as directed by the manufacturer. Each sonicated sample was centrifuged at 13,000 g for 10 min. Each supernatant was collected in a 1.5-mL tube and boiled with 20 mM dithiothreitol (final concentration) at 95° C. for 10 min. Then, the solution was cooled to room temperature and alkylated with 40 mM iodoacetamide in the dark for 30 min. Subsequently, the sodium dodecyl sulfate lysate was added to 12% aqueous phosphoric acid (1:10 dilution, yielding a final concentration of 1.2% phosphoric acid) and seven volumes of binding buffer (90% aqueous methanol with a final concentration of 100 mM triethylammonium bicarbonate, TEAB; pH 7.1). After gentle mixing, the protein solution was loaded onto the S-Trap filter, spun at 3,000 g for 1 min, collected using flow-through, and reloaded onto a filter. This step was repeated two times, and the filter was washed three times with 400 μL of binding buffer. Finally, 10 μg of trypsin (Promega) and 125 μL of digestion buffer (50 mM TEAB) were added to the filter at 1:25 w/w and digested at 37° C. for 16 h. To elute the digested peptides, three step-wise buffers were applied, with 80 μL of each peptide repeated once; these buffers included 50 mM TEAB, 0.2% formic acid in water, and 50% acetonitrile/0.2% formic acid in water. The peptide solution was pooled, lyophilized, and desalted in accordance with the protocol of the Pierce™ Peptide Desalting Spin Column (Thermo Fisher Scientific, Waltham, MA, USA).

### TMT 11-Plex Labeling

To compare data between samples, multiplexing was used with four sets of TMT11-plexes for 8 normal tissues and 16 tumor tissues. A pooled common control was constructed as a reference to facilitate combinations of data for multiple sets of TMT11-plexes. The control consisted of equal weights of total peptides from each of the samples used in the experiment. Each TMT11-plex consisted of three aliquots of the relevant common control at a ratio of 0.5:1:2, along with 8 individual samples. In total, 100 μg of desalted peptides were measured using the Pierce™ Quantitative Fluorometric Peptide Assay kit, in accordance with the manufacturer's instructions (Thermo Fisher Scientific). The desalted and dried peptides were re-dissolved in 100 mM TEAB (100 μL) with TMT 11-plex reagents, in accordance with the manufacturer's instructions (Thermo Fisher Scientific). Next, 0.8 mg of TMT reagent (41 μL) was added to each sample, mixed, and incubated at room temperature for 1 h. The reactions were quenched using 8 μL of 5% hydroxylamine (Thermo Fisher Scientific) and incubated at room temperature for 15 min. The labeled samples (25-100 μg) were combined, dried, and desalted using Pierce™ Peptide Desalting Spin Columns (Thermo Fisher Scientific). The eluates were dried and stored at −80° C.

### High pH Reversed-Phase Fractionation

The TMT-labeled peptides were fractionated using a Shimadzu HPLC system that consisted of a binary pump, an autosampler, a degasser, a variable wave detector, and a fraction collector. High pH reversed-phase fractionation was performed using a 4.6×150 mm Waters XBridge® BEH C18 column (diameter, 2.5 μm). Mobile phase A consisted of 5 mM ammonium formate in 100% water, whereas mobile phase B consisted of 5 mM ammonium formate in 95% acetonitrile. Sample separation was accomplished using the following linear gradient: 5% B for 15 min, from 5% to 15% B over 5 min, from 15% to 40% B over 30 min, 40% B for 5 min, from 40% to 95% B over 4 min, 95% B for 4 min, from 95% to 5% B over 1 min, and 5% B for an additional 9 min. Time-dependent fractions were collected from 21 to 61 min for a total of 40 fractions, yielding approximately 1 mL/fraction. The variable wave detector was monitored at 214 nm. After collection, 40 fractions were combined into 20 fractions by blending fractions (e.g., 1 and 21; 2 and 22; 3 and 23). Each fraction was dissolved in 200 μL water/formic acid (99.9:0.1, v: v) for LC-MS/MS analysis.

### Nano LC-Electrospray Ionization-MS/MS Analysis

A nano-flow ultra-high-performance liquid chromatography (UHPLC) system (UltiMate 3000 RSLCnano System; Thermo Fisher Scientific) coupled to the Orbitrap Eclipse™ Tribrid™ mass spectrometer (Thermo Fisher Scientific) was used for proteome analyses. Fractionated peptides were injected and separated on EASY-Spray PepMap™ RSLC C18 Column ES803A (2 μm, 100 Å, 75 μm×50 cm; Thermo Fisher Scientific) operated at 45° C. A gradient from 5% to 95% mobile phase B was applied over 140 min with a flow rate of 250 nL/min, using mobile phases A (water/formic acid, 99.9:0.1, v:v) and B (acetonitrile/formic acid, 99.9:0.1, v: v). The electrospray ionization voltage was 1800-1900 V, and the ion transfer tube temperature was 275° C.

UHPLC-MS/MS data were acquired using a data-dependent top-speed mode comprising a full scan to maximize the number of MS2 scans during the 3 s of cycle time. The full scan (MS1) was detected using the Orbitrap analyzer at a resolution of 120 K, with a mass range of 400-2000 m/z. The automatic gain control target mode was “standard,” the maximum injection time mode was “auto,” the charge states were set at 2-6, and a dynamic exclusion window was set at 30 s. The second scan (MS2) was collided by the higher-energy C-trap dissociation (HCD) mode. The HCD spectra were detected using the Orbitrap analyzer at a resolution of 30 K with 37% fixed collision energy for isobaric labeled peptides. The maximum injection time mode was “auto,” the isolation window was 0.7, the automatic gain control target mode was “standard,” the first mass was fixed at 110, and the mode was Turbo TMT.

### Data Processing

For proteomics analysis, raw files were converted to MS (.ms1) and MS2 (.ms2) files using RawConverter (The Scripps Research Institute, La Jolla, CA, USA). Proteome search and database generation were conducted using IP2 (Integrated Platform for mass spectrometry data analysis, Bruker). Proteome results were analyzed using ProLuCID, DTASelect2, and Census. The database for analysis was generated using the UniProt human proteome database (20,645 entries, updated on Jan. 1, 2020). The following IP2 parameters were used: precursor and fragment mass tolerance, 50 ppm; enzyme, trypsin; miscleavages, ≤2; static modifications, 57.0215 Da added at cysteine, 229.1629 Da added at lysine and N-terminal; differential modifications, 15.9949 Da added at methionine; and minimum number of peptides per protein, 2. Pooled spectral files from all 20 fractions were compared with both normal and reversed databases using the same parameters. For peptide validation, the false positive rate was 0.01 of the spectrum level. TMT reporter ion analysis was conducted using Census software, with a mass tolerance of 20 ppm. Similar data processing methodology was applied for proteomics analysis of non-decellularized native CRC samples using published raw data in the CPTAC Data Portal (https://cptac-data-portal.georgetown.edu/study-summary/S037).

### Enhancement of Spectral Quantitative Accuracy Using Pooled Internal Standards

Three TMT channels were used as internal references with a pooled common control, which represented pooled peptides of equal amounts from all samples; this approach allowed the assessment of intra-and inter-batch variance, while enhancing quantitative accuracy. The pooled common control was labeled with TMT 130N, 131C, and 131N reagents at a ratio of 0.5:1:2; these reagents served as reference channels. Using the central limit theorem, the log2 ratio of the three reference channels (log2 TMT channels 131N/131C, 131C/130N, and 131N/130N) for all peptides measured in the proteomic analysis was expected to fit a standard Gaussian distribution with near one (131N/131C), near one (131C/130N), and near two (131N/130N), respectively; this method can be used to assess variations in technical replications. We implemented a filtering criterion based on the multidimensional significance offered by Perseus. The Benjamini-Hochberg false discovery rate was used for truncation, with a threshold value of 0.05. Using these criteria, the outlier spectrum was filtered to enhance quantitative accuracy.

### Normalization of Protein Abundance

Because of differences in sample handling and laboratory environments, there were systematic and sample-specific biases in the quantification of protein abundance. To eliminate these effects, we calculated the median of log2-transformed peptide abundance; column values were subtracted from median values to achieve a common median of 0. Then, we calculated the average of the median values, re-added them to the zero-centered column, and transformed the re-centered value using the y=2\hat{ }(x) function. For inter-sample intensity normalization, the relative intensity value of each protein was calculated through division of the intensity values of the proteins in each sample by the original intensity value of the R2 column, which was used as the reference for other samples. Then, the final normalized intensity values were calculated through multiplication of the relative intensity value of each protein by the average normalized intensity value of the R2 column. The normalized value was transformed using the y=2\hat{ }(x) function. The abundance values were used for further proteome analysis.

### Statistical Analysis for TMT Proteomics

TMT-based proteomics data were used to perform hierarchical clustering, PCA, and DEP analysis. For hierarchical clustering, the normalized intensity values were scaled and clustered with the matrisome protein data based on the Euclidean distance in Perseus software. For PCA, only normalized intensity values of matrisome proteins were used. DEPs between the tumor and normal tissues were determined using Welch's t-test with Benjamini-Hochberg correction. DEPs with foldchange >√2 and adjusted p<0.01 were selected. GSEA of DEPs was performed using gene sets provided by Metascape and p-values were used to identify enriched genes.

scRNA-Seq and Data Analysis

scRNA-Seq analysis of CRC tissues was performed using published data from a previous study. Briefly, single-cell CRC dissociates of Samsung Medical Center cohorts were collected and a barcoded sequencing library was generated in accordance with the manufacturer's instructions. Specific parameters, reagent kits, and pipelines for sequencing were used as previously described. Processed scRNA-Seq data and metadata, including information related to cell annotation, were used for the Samsung Medical Center cohorts. Six global cell types (epithelial, stromal, B, T, myeloid, and mast) and 25 subdivisions were used for further analysis. Normal fibroblasts and tumor fibroblasts were defined on the basis of tissue source for the analyzed single cells.

The cellular origins of DEPs were identified using the average expression levels of cell types. Cell type-specific genes were defined using the FindAllMarkers function in the Seurat package; an adjusted p<0.01 was used as a threshold to determine whether the gene expression was cell type-specific. The cell type-specific average expression levels were determined using the AverageExpression function in the Seurat package; the cell type with the highest average expression level was regarded as the cellular origin of the gene.

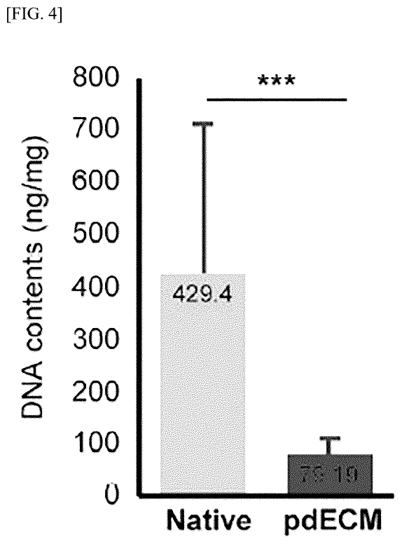

To define the TAM and NAM, we used only fibroblasts that were previously annotated with the fibroblast cell type. The gene expression patterns of fibroblasts were normalized and clustered by 1) performing linear dimensional reduction using the RunPCA function in the Seurat package with all matrisome genes regarded as features, 2) using the FindNeighbors function in the Seurat package with the parameter dims=1:20, 3) using the FindClusters function in the Seurat package with the parameter resolution=0.5, and 4) using the RunTSNE function in the Seurat package with the parameter dims=1:20 to plot fibroblasts in the dimensional space. Then, clusters that were specific condition-dominant (i.e., normal vs. tumor) (>90% of cells in a cluster had the same condition) and clusters that consisted of cells from >2 patients were re-clustered into metaclusters (FIG. 4a; clusters 0, 3, 7: tumorfibroblast metacluster; clusters 1, 2, 4, 5, 6: normal-fibroblast metacluster). Tumor-associated and normal-associated marker genes between the two metaclusters were defined using the FindMarkers function in Seurat with adjusted p<0.01. The TAM and NAM were defined by calculating the foldchange of average protein intensity between the normal and tumor groups. Among the TAM marker genes, when the average intensity of the protein was higher in the tumor group, the protein was included in the TAM. Similarly, among the NAM marker genes, when the average intensity of the protein was higher in the normal group, the protein was included in the NAM.

### Bulk Tissue RNA-Seq and Bioinformatics Analysis

The collected CRC tissues were maintained in TRIzol reagent for bulk tissue RNA-Seq. The indexed cDNA sequencing libraries were prepared from RNA samples using the TruSeq Stranded mRNA LT Sample Prep Kit. Quality control analyses of RNA integrity number and rRNA ratio were performed using the 2200 TapeStation. The indexed libraries were prepared as equimolar pools and sequenced on the NovaSeq 6000 to generate a minimum of 60 million paired-end reads per sample library. The raw Illumina sequence data were demultiplexed and converted to fastq files. Then, the adaptor and lowquality sequences were trimmed. The mRNA sequencing reads were mapped to Homo sapiens genome assembly GRCh37 from the Genome Reference Consortium by HISAT2 (version 2.1.0). Mapped reads were assembled with known genes and quantified in terms of read counts and sample normalized values, such as fragments per kilobase of transcript per million mapped reads and transcripts per million mapped reads (TPM), using StringTie (version 2.1.3b). TCGA, COAD, and READ gene expression datasets and a clinical dataset from the TCGAbiolinks package were collected for analyses of CMS-specific gene expression patterns. After the gene expression information had been downloaded from the Illumina platform, the raw counts were converted to normalized TPM values. Clinical information (e.g., the parameters days_to_last_follow_up, death_days_to, and new_tumor_event) was collected and used for analysis of PFS. In total, 612 tumor samples and 51 normal samples were analyzed. For CMS classification, the CMSclassifier package was used to identify the CMS of collected CRC tissues and TCGA samples. Gene expression values were used after log2-transformation of TPM data and summed to the nearest 0.001. The NearestCMS values and CMS4 probability were calculated using the random forest algorithm. Samples with an ambiguous CMS classification, where the assigned subtype did not constitute a single subtype, were not used for further analysis. For identification of CMS4-enriched matrisome genes, normalized TPM data of TCGA samples were subjected to GSEA. In total, 38 matrisome markers defined as TAM or NAM were used as the gene set. Based on enrichment scores derived from GSEA, core enrichment genes were defined as CMS4-enriched TAM/NAM markers. The expression patterns of specific gene sets in each TCGA sample were evaluated using ssGSEA. Normalized TPM data of CMS-classified TCGA samples were preprocessed. The ssGSEA scores for gene sets associated with EMT (MSigDB M5930) and the TGFβ response in fibroblasts (gene sets from PMID: 23153532), as well as customized gene sets that consisted of 29 CMS4-enriched TAM/NAM molecules in GSEA and 10 markers that were clinically significant, were calculated using the ssGSEAprojection package in the GenePattern web-based tool. The calculated scores were log2-transformed and normalized to determine correlations among ssGSEA scores.

### Tissue Immunohistochemistry (IHC)

Tissue immunohistochemical analysis was performed on 4-nm formalin-fixed, paraffin-embedded (FFPE) tumor tissue slide sections. The slides were deparaffinized in xylene and absolute alcohol, then rehydrated through a descending alcohol gradient ending in water. For antigen retrieval, the slides immersed in 10 mM sodium citrate buffer (pH 6.0) were heated in a microwave oven for 10 minutes, followed by blocking of endogenous peroxidase activity using 3% hydrogen peroxide dissolved in methanol for 30 minutes. After rinsing in TBS, potential nonspecific binding was blocked by incubating the slides for 30 minutes in 5% BSA (for HAPLN1) or 10% BSA (for COL12A1 and THBS2). Primary antibodies—HAPLN1 (goat anti-human polyclonal Ab, 1:400 dilution, Biotechne, MN, USA), COL12A1 (rabbit anti-human polyclonal Ab, 1:200 dilution, Sigma-Aldrich, MA, USA), or THBS2 (mouse anti-human monoclonal Ab, 1:1000 dilution, Invitrogen, MA, USA)—were incubated at 4° C. overnight. After washing the slides with TBS, appropriate secondary antibodies diluted 1:200 in TBS were applied using the Vectastain ABC kit (Vector Laboratories, CA, USA) for 30 minutes, and detection was performed using DAB solution (Dako, CA, USA). The sections were counterstained with hematoxylin, dehydrated through an ascending ethanol series, and mounted under a coverslip using synthetic mountant (Thermo Fisher Scientific, MA, USA).

### Results

### Quantitative Proteomic Analysis of Decellularized Patient-Derived Tissue

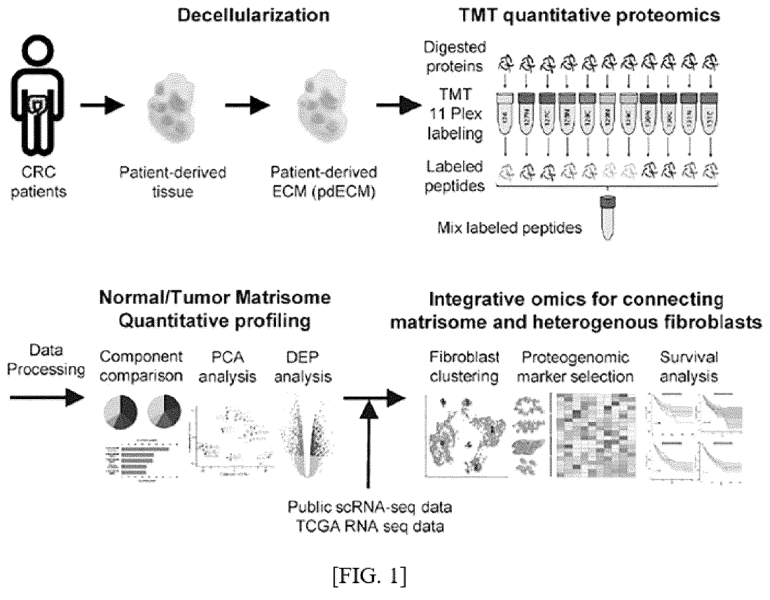

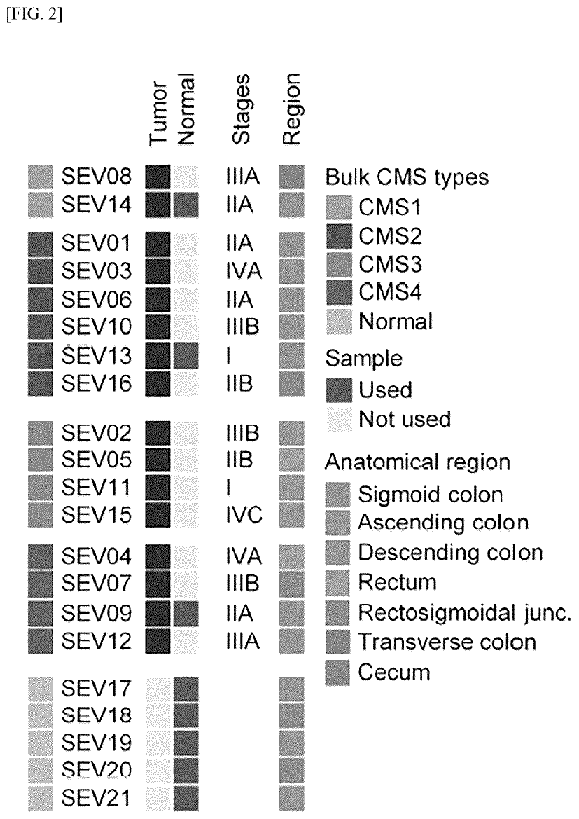

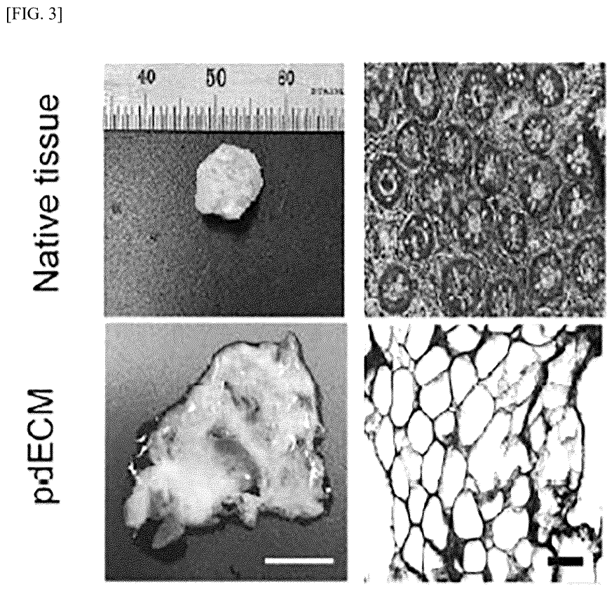

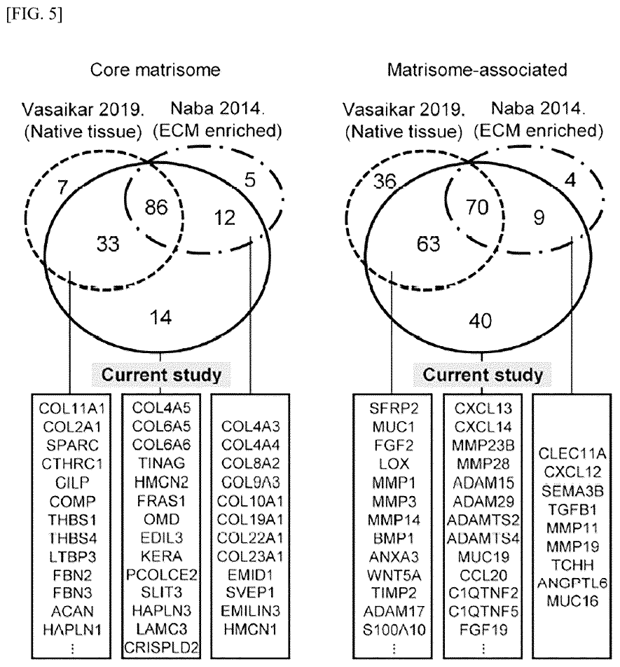

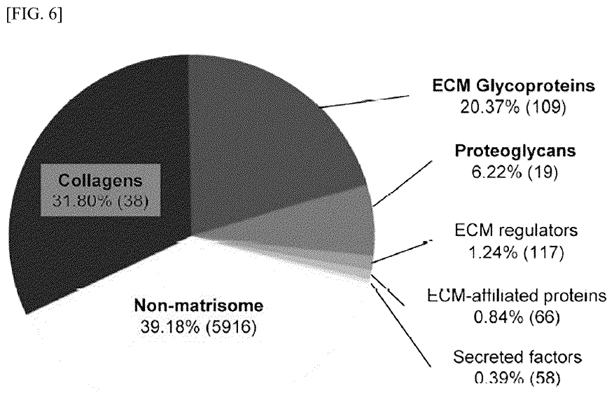

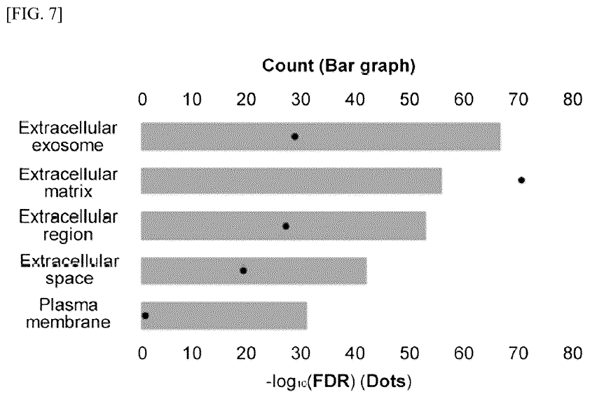

To investigate the composition of ECM proteins in CRC, we utilized detergent-based decellularization to enrich ECM proteins from human tumor tissues. In the present invention, the series of processes for decellularizing and analyzing colorectal cancer tissues is illustrated schematically (FIG. 1). Normal adjacent and tumor tissue were acquired surgically from 22 patients with CRC. FIG. 1b provides a summary of the clinical data, tumor stage, location, and consensus molecular subtype (CMS) for each patient (FIG. 2). Hematoxylin and eosin stain (H&E) and DNA quantification confirmed the enrichment of ECM proteins (FIG. 3). We confirmed that decellularization caused a substantial loss of nuclei, a reduction in genomic DNA, and the preservation of ECM architecture (FIG. 4). For comparative proteomics analysis of ECM-enriched samples, liquid chromatography-mass spectrometry (LC-MS)/MS analysis on an isobaric tandem mass tag (TMT) was utilized (see Methods for details). In total, 24 dried mass-matched patient-derived ECM (pdECM) samples from normal and tumor tissues were serially processed to perform four TMT-11plex sets. A sample prepared by pooling all samples was used as a reference for quantitative analysis; they were also used to calculate the fold changes of proteins between normal and tumor samples. In total, we identified 6,323 proteins with no “NA” values in all samples of one set. According to the Human Matrisome Database, 407 of these proteins are matrisome proteins (collagens [COLs], proteoglycans [PGs], and ECM glycoproteins [GPs]) and matrisome-associated proteins.7 Furthermore, 145 of 166 core matrisome proteins and 182 of 241 matrisome-associated proteins were detected in at least all sets with tumor samples or all sets with normal samples. When compared with previous studies (Vasaikar, S. et al. Proteogenomic analysis of human colon cancer reveals new therapeutic opportunities. Cell 177, 1035-1049.e1019 (2019), and Naba, A. et al. Extracellular matrix signatures of human primary metastatic colon cancers and their metastases to liver. BMC Cancer 14, 1-12 (2014)), 98 core matrisome proteins and 79 matrisome-associated proteins were identified in those studies, whereas in the present study, only 47 core matrisome proteins and 103 matrisome-associated proteins were detected (FIG. 5). Importantly, miscellaneous ECM glycoproteins were detected only in our TMT-based platform, including fibrillin 3 (FBN3), nidogen 2 (NID2), ABI family member 3 binding protein (ABI3BP), laminin subunit alpha 3 (LAMA3), and thrombospondin 1 (THBS1); these glycoproteins are universally present in the ECM of normal colon and tumor samples.9 Our results suggest that the our TMT-based quantitative proteomics analysis of ECM-enriched CRC tissues can provide the largest datasets of human colon matrisome. Additionally, compared with native tissue proteomics data, 10 the core and associated matrisome components benefit from enrichment. Notably, 14 core matrisome proteins, which are mainly insoluble and cross-linked, were detected only by our platform. To investigate the relative percent composition (RPC) of detected matrisome proteins in pdECM samples against native tissue, the RPC of each protein was calculated by dividing each protein intensity by the total sum of all protein intensities and expressed with percentage. The RPC of each category of matrisome was determined by summing all the RPCs of protein corresponding to each category of 5 matrisome. As a result, the total RPC of matrisome proteins was substantially higher in the decellularized tissue samples than in the non-decellularized native tissue (FIG. 6). The RPCs of core matrisome, which includes COL, GPs, and PGs, corresponds for 58.67%, which is nearly sevenfold higher than the RPC in native tissues (8.92%). Furthermore, the RPC of non-matrisome proteins agreed with the RPC that measured in other decellularization studies (32-41%). To identify the linked cellular components, a Gene Ontology (GO) analysis of the top 100 proteins with the greatest intensities was undertaken. ECM-associated proteins were shown to be enriched in pdECM, but nuclear and intracellular proteins were not. In contrast, cytosolic and nuclear proteins were shown to be more abundant in non-decellularized tissue (FIG. 7). These findings suggest that our ECM-protein enrichment approach enables detailed identification of matrisome components by LC-MS/MS.

Quantitative ECM Proteomics Analysis of pdECM Samples From Normal and Tumor Tissues

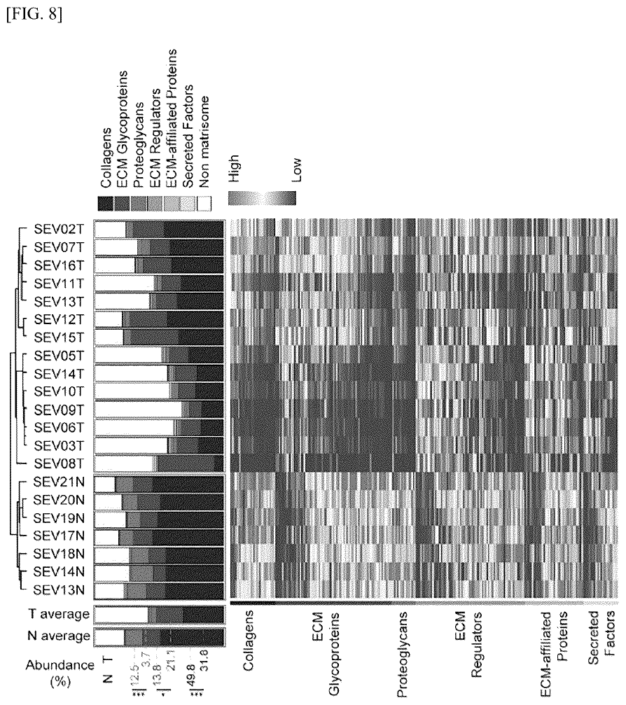

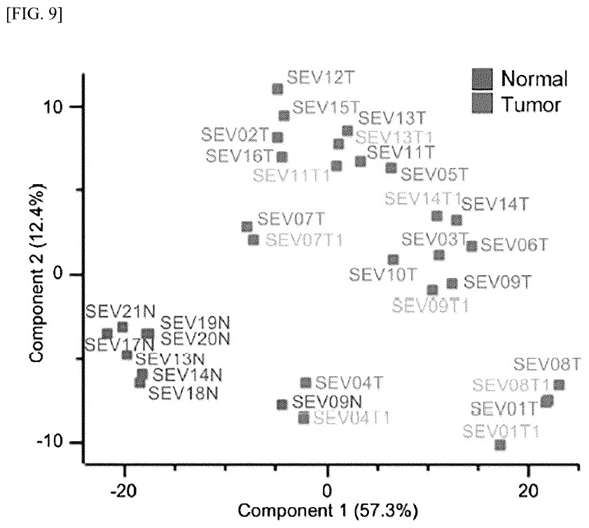

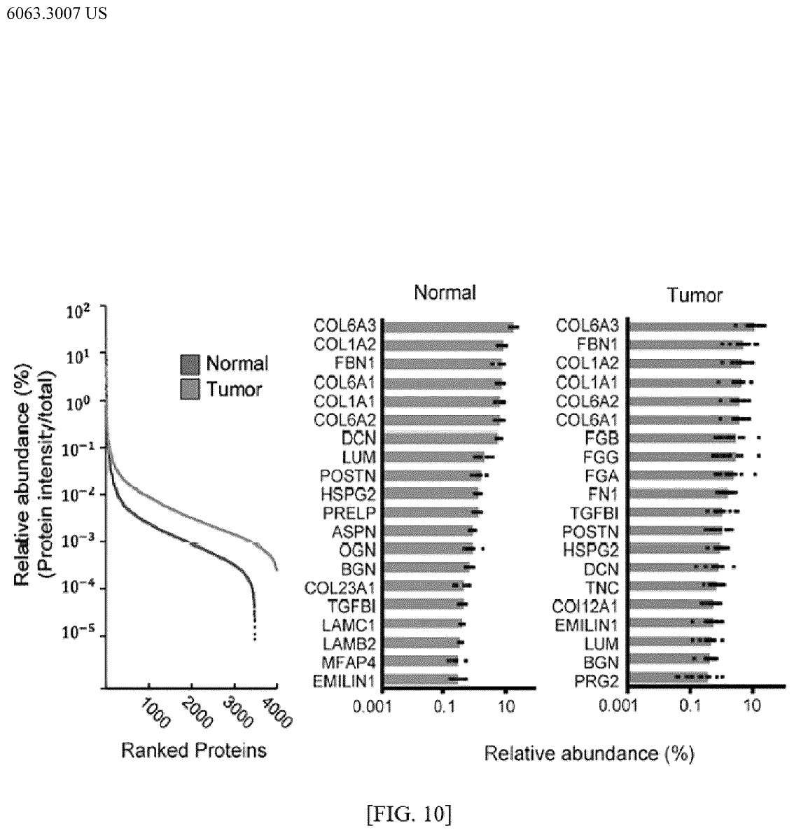

To examine the matrisome components in pdECM samples of normal and tumor tissues, we compared their quantitative proteomic profiles. In both normal and tumor tissues, 123 of 255 matrisome proteins were identified as core matrisome proteins. Hierarchical clustering with a matrisome profile revealed that, across multiple patients, all normal samples clustered together and demonstrated similar proteomic expression patterns. The matrisome in tumor samples was highly heterogeneous and significantly differed from the normal samples. The RPC of GPs was significantly increased in tumor tissues. Additionally, the RPC of matrisome-associated proteins and secreted factors were increased in tumor tissues. In contrast, the RPC of COLs was significantly reduced (by 27.8%). Consistent with the global changes in collagens, the RPC of PGs decreased from 12.3% to 3.5%, although some proteins had higher levels in tumor samples than in normal samples (FIG. 8). Principal component analysis (PCA) revealed a difference between the normal and tumor groups, but no differences among normal samples. In contrast, PCA showed greater distances among tumor samples. Replicate samples were located near each other in the PCA plot, which confirmed the reproducibility of the proteomics analysis. The calculated distance coefficients between the normal samples and tumor samples indicated that normal tissues were generally similar and tumor tissues were generally heterogeneous (FIG. 9). Some samples were excluded from analysis because of factors that could have affected ECM composition, such as chemotherapy, perforation, or stent insertion (SEV01T: perforation; SEV04T: chemotherapy; SEV09N: stent insertion). The excluded samples showed protein expression patterns distinct from other samples; thus, they were analyzed by hierarchical clustering and PCA. The results indicated that the ECM composition was associated with the pathological and histological features of each clinical sample. Next, we compared the abundant proteins in pdECM samples of normal and tumor tissues. We ranked the detected proteins according to RPC of each protein of each condition (Normal/Tumor). The results showed that 81 and 855 protein components covered 90% of RPC in pdECM samples of normal and tumor tissues, respectively (FIG. 10). This result indicated that the protein components of normal tissues were more uniformly distributed among the samples, compared with the protein components of tumor tissues. The top six most abundant proteins from each matrisome in both groups had a similar composition and abundance. However, the top 20 matrisome proteins with the highest intensities differed between normal and tumor tissues, which is consistent with previous studies of the matrisome in CRC tissues. Among the 20 proteins, 13 were highly expressed in both normal and tumor tissues. The top six most abundant proteins encoded by COL6A1/2/3, COL1A1/2, and FBN1 detected in both normal and tumor tissues. Three type VI COLs (encoded by COL6A1, COL6A2, and COL6A3) and two type I COLs (encoded by COL1A1 and COL1A2) constituted a significant proportion of the human colon ECM in normal (55.6%) and tumor (31.8%) tissues. Decorin (DCN) and lumican (LUM), which are involved in the regulation of COL fibril assembly and stability, had high abundances in both normal and tumor tissues; their levels were much higher in normal tissue than in tumor tissue. GPs, such as the fibrinogen family (FGA, FGB, and FGG), fibronectin (FN1), transforming growth factor beta-induced protein (TGFβI), and tenascin-C (TNC), had an increased presence in tumor tissues. Notably, the expression profiles of COLs and PGs were inversely correlated with the levels of the metzincin family of metalloproteinases, including two matrix metalloproteinases (MMPs; MMP9 and MMP14) and two disintegrin and metalloproteinases (ADAMs; ADAM9 and ADAM10); these metalloproteinases play key roles in ECM remodeling that involve the proteolytic degradation of ECM components. 15 Our study identified the major components of ECM and revealed significant changes in the abundance and organization of ECM in CRC tissues.

Differentially Expressed Matrisome Proteins in pdECM Samples of Normal and Tumor Tissues

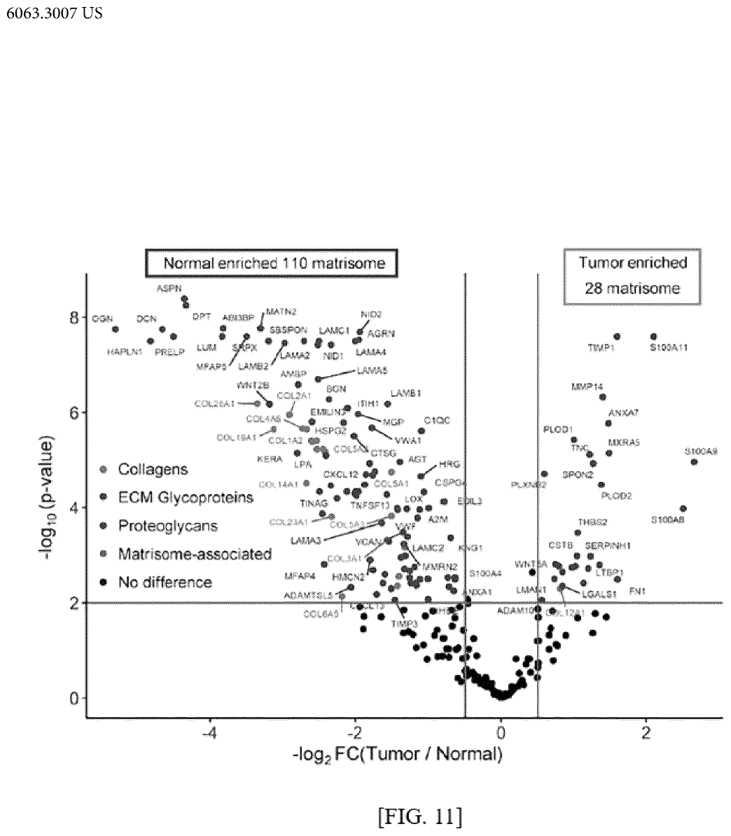

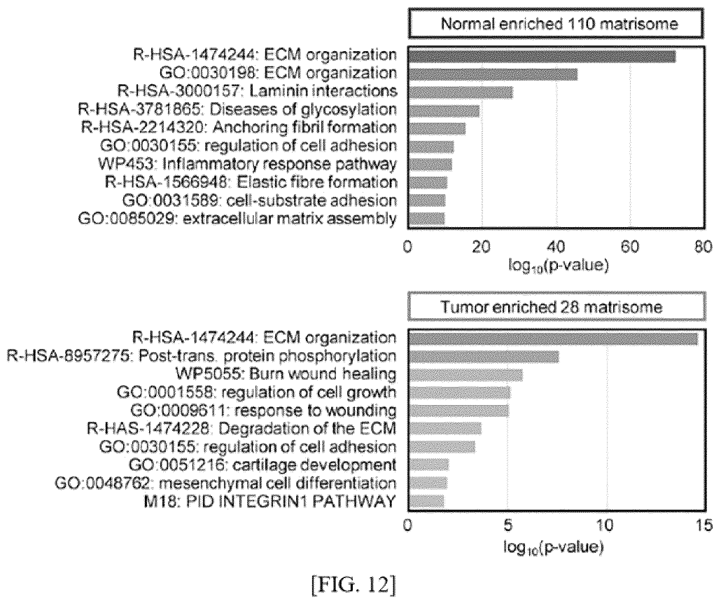

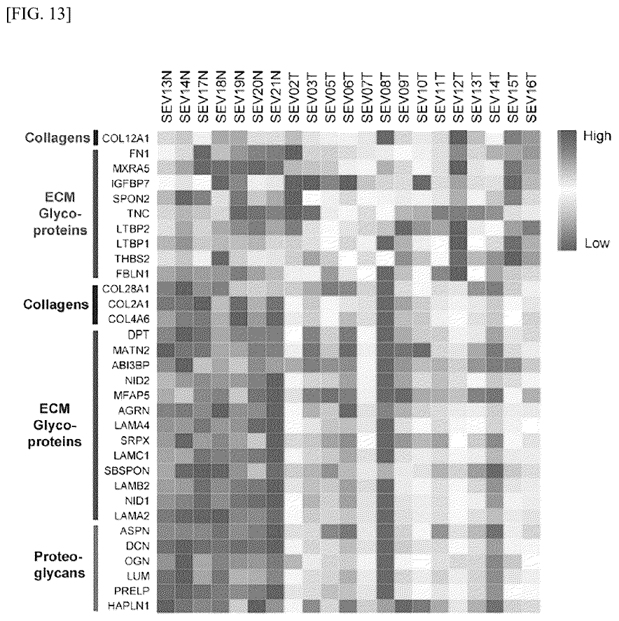

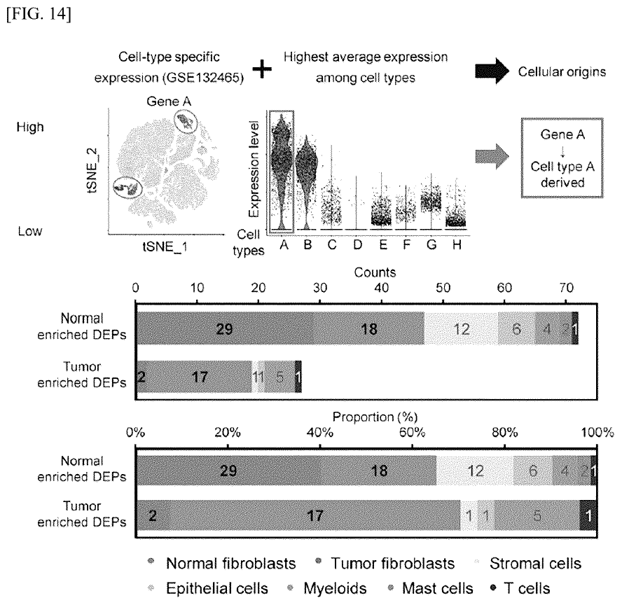

To identify compositional changes in the ECM microenvironment, we compared the matrisomes of normal and tumor tissues by differentially expressed protein (DEP) analysis. For each protein, we calculated the fold change between normal and tumor tissues along with the adjusted p-value according to Welch's t-test, and summarized the matrisome DEPs in a volcano plot (FIG. 11). As a result, 110 and 28 matrisome proteins were enriched in pdECM samples from normal and tumor tissues, respectively. Functional gene-set analysis identified wound healing and ECM degradation, which are the main biological terms associated with fibroblast activation (FIG. 12). The heatmap of selected core matrisome proteins showed significantly upregulated proteins in normal and tumor tissues (FIG. 13). In total, 32 core matrisome proteins were selected, including all tumor-enriched proteins, three normal-enriched COLs with the lowest p-values, and matrisome proteins with −log10(p-value)>7. Among the tumor-enriched DEPs, significant differences in protein abundance were detected for the GPs group, except for type XII COL alpha 1 chain (COL12A1). Consistent with our proteomics data, COL12A1 was upregulated in various cancers, including CRC. COL12A1, which encodes the α1 chain of collagen XII, has been reported as a novel stromal marker with robust expression in the desmoplastic stroma of CRC tissues. Among the tumor-enriched GPs, matrix-remodeling associated protein 5 (MXRA5) had the greatest statistical significance (p=7.13×10⁻⁶); this finding is consistent with the results of a previous study in which MXRA5 was aberrantly expressed in CRC tissues. In addition, multiple COLs, GPs, and PGs were abundantly present in normal tissues. In particular, PGs in the small leucine repeat proteoglycans (SLRPs) family (e.g., DCN, LUM, ASPN, and OGN) were most significantly enriched in normal ECM. The upregulation of proteinases (i.e., MMPs and ADAMTS) in tumor tissues supports that proteases digestion could induce the depletion of extracellular SLRPs under pathophysiological conditions. Furthermore, because SLRPs regulate COL fibril organization and stability, SLRP depletion may cause ECM dysfunction by interfering with COL network stability and accelerating COL degradation in CRCs. We next evaluated the cellular origin of matrisome proteins to identify stromal-centric remodeling in the CRC microenvironment. For this purpose, we re-analyzed public single-cell RNA sequencing (scRNA-Seq) data of CRC tissues to investigate the cellular origins of matrisome proteins. Individual DEPs were considered “specific cell-derived” when the gene encoding DEPs were significantly differentially expressed in a specific cell subtype (adjusted p<0.01). We assigned the cellular origins of 138 DEPs based on the most significantly expressed subtypes within the seven cell subtypes, including normal-derived fibroblasts, tumor-derived fibroblasts, other stromal cells, epithelial cells, myeloid cells, mast cells, and T cells. Among these 138 DEPs, 99 matrisome proteins were regarded as specific cell-derived proteins (FIG. 14). Among normal-enriched and tumor-enriched DEPs, 47 and 19 matrisome proteins were fibroblast-derived, respectively. In comparison, only seven proteins were derived from epithelial cells: laminin subunit alpha 3 (LAMA3), beta 3 (LAMB3), gamma 2 (LAMC2), secretory leukocyte peptidase inhibitor (SLPI), semaphorin 3B (SEMA3B), mucin 5B (MUC5B), and plexin B2 (PLXNB2). Although the protein levels were not consistently correlated with gene transcript levels, most tumor-enriched DEPs corresponded to the tumor tissue-derived fibroblasts, supporting the notion that cancer-associated fibroblasts (CAFs) are major determinants of ECM remodeling in the TME. Therefore, we further studied the molecular features of fibroblasts involved in tumorous ECM to achieve a comprehensive understanding of ECM-centric microenvironment remodeling in CRCs.

### Integrative Omics Analysis for the Identification of Normal-Associated and Tumor-Associated Matrisome Proteins

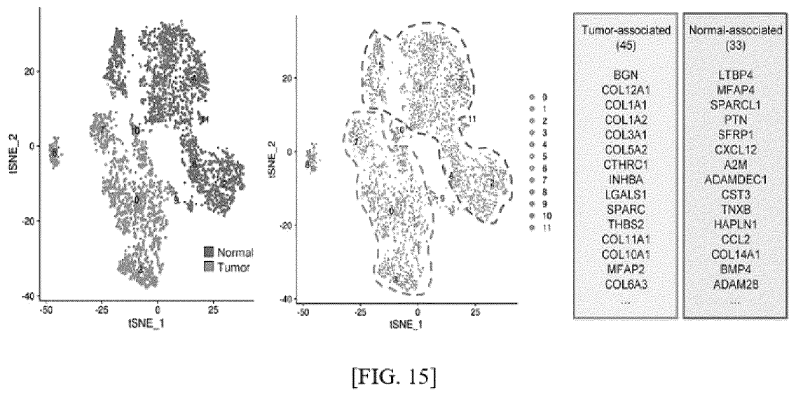

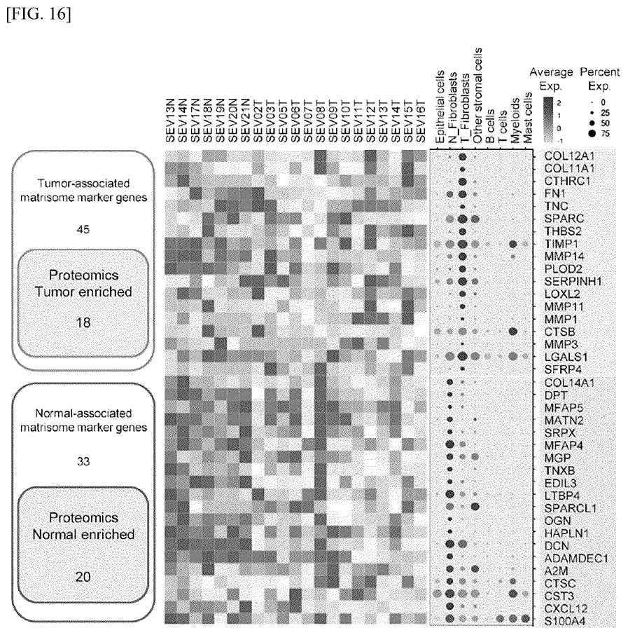

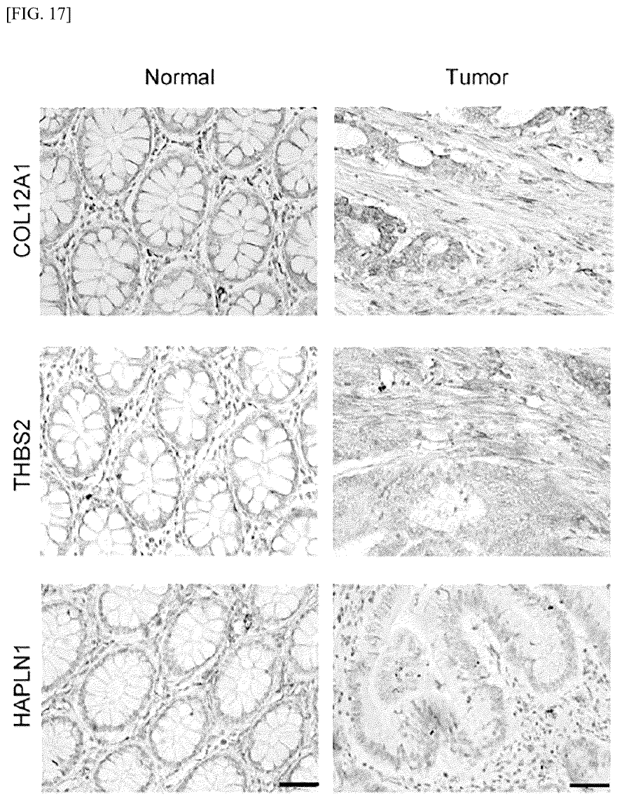

To explore the functional contribution of fibroblasts to ECM remodeling in CRC, we re-analyzed 3,462 fibroblasts from a published scRNA-Seq dataset. We defined the normal and tumor metaclusters as normal-derived and tumor tissue-derived fibroblasts, respectively; we identified the differentially expressed genes in each metacluster (45 genes in normal tissue and 33 genes in tumor tissue) (FIG. 15). Then, we analyzed these molecules at the protein level using our proteomic dataset. Among the proteins that were encoded by the 45 matrisome genes upregulated in tumors, 18 were enriched in the tumor tissue-derived matrisome marker; among the proteins that were encoded by the 33 matrisome marker upregulated in normal tissues, 20 were enriched in the normal tissue-derived matrisome marker. Therefore, we defined 18 tumor-associated matrisome (TAM) proteins and 20 normal-associated matrisome (NAM) proteins as stromal markers in CRC (FIG. 16). Among the 38 matrisome proteins that were quantifiable in our proteomics data, most NAMs, excluding SPARC-like protein-1 (SPARCL1), were upregulated in normal tissues. In contrast, TAM proteins exhibited nonuniform upregulated expression in the tumor samples. Dot plot analysis of the scRNA-Seq data showed that most TAM and NAM proteins were associated with tumor-derived and normal-derived fibroblasts, respectively. In particular, SPARCL1 exhibited patient-specific expression at the protein level and enriched expression at the transcript level in other stromal cells, but not in normal-derived fibroblasts. This result is consistent with previous findings that SPARCL1 is preferentially expressed by endothelial cells in human CRC tissues. Among TAM proteins, COL12A1, collagen triple helix repeat containing 1 (CTHRC1), THBS2, MMP14, and procollagen-lysine-2-oxoglutarate 5-dioxygenase 2 (PLOD2) were tumor-derived fibroblast-specific proteins. More than 70% of tumor-derived fibroblasts exhibited upregulation of gene transcripts compared with other stromal cells, indicating that TAM proteins in CRC tissue are mainly produced by CAFs. Among the TAMs and NAMs, three proteins were selected for validation of tissue localization: COL12A1, THBS2, and hyaluronan and proteoglycan link protein-1 (HAPLN1). scRNA-Seq data indicated that these proteins were predominantly expressed by fibroblasts. Immunohistochemistry revealed findings similar to our proteomics results (FIG. 17). Normal mucosa exhibited weak staining of COL12A1 and THBS2; conversely, tumor tissue exhibited strong staining of these proteins, and the staining was almost exclusively localized to stromal cells. Furthermore, HAPLN1 staining was observed only in the stroma of normal mucosa, whereas most epithelial cells did not exhibit HAPLN1 staining. HAPLN1 is an ECM protein that maintains the ECM integrity by stabilizing other ECM proteins. Our results are consistent with a previous report that HAPLN1 exhibits decreased protein expression in CRC, presumably because the loss of HAPLN1 in CRC results from fibroblast remodeling (e.g., loss of HAPLN1-expressing normal fibroblasts). Our data suggests that the transcriptomic features of fibroblasts reflect compositional remodeling of the tumorous ECM microenvironment.

### CMS4-Specific Normal/Tumor-Associated Matrisome Markers and Clinical Relevance

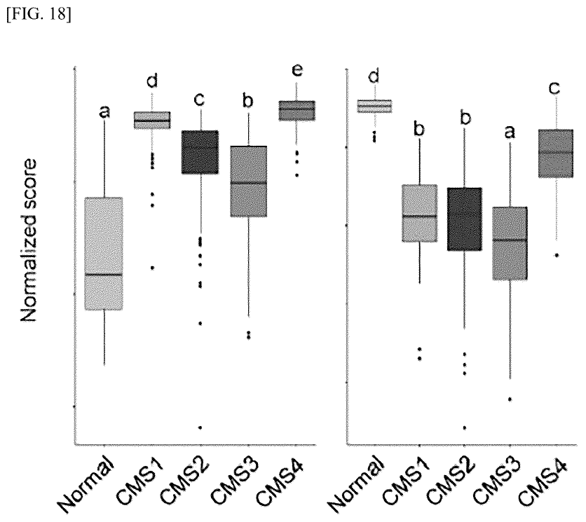

The consensus molecular subtype (CMS), a classification based on transcriptional profiles, was recently developed to emphasize the importance of the ECM microenvironment in CRC. The CMS describes four CRC subtypes, among which the mesenchymal subtype or CMS4 group is characterized by extensive stromal infiltration (mostly activated fibroblasts) and ECM organization. Recent studies have demonstrated that CAFs in CRCs are composed of distinct fibroblast populations and significantly enriched in the CMS4 subtype compared with the other subtypes. Therefore, we sought to compare ECM features between the myofibroblast-enriched CMS4 subtype and other subtypes. We performed single-sample gene set enrichment analysis (ssGSEA) with The Cancer Genome Atlas (TCGA)-Colon Adenocarcinoma (COAD)/Rectal Adenocarcinoma (READ) expression data sets to calculate the expression patterns of TAM and NAM. The TAM ssGSEA scores were significantly higher in the stroma-enriched molecular subtype (CMS4) than in other cell types. The NAM scores were higher in normal tissues than in tumor tissues, and the scores varied according to tumor tissues (FIG. 18). In particular, the levels of NAM were higher in the CMS4 subtype than in other subtypes, but the transcript levels of NAM were slightly lower in the CMS4 subtype than in normal tissues. Overall, fibroblasts from CMS4 showed increased transcript levels of most ECM genes, which is consistent with the molecular features of ECM organization and stromal invasion.

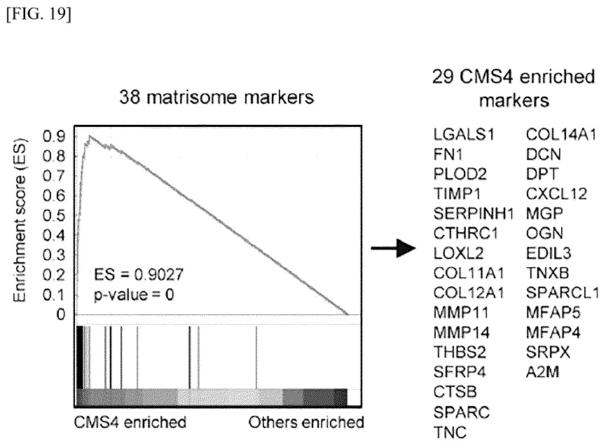

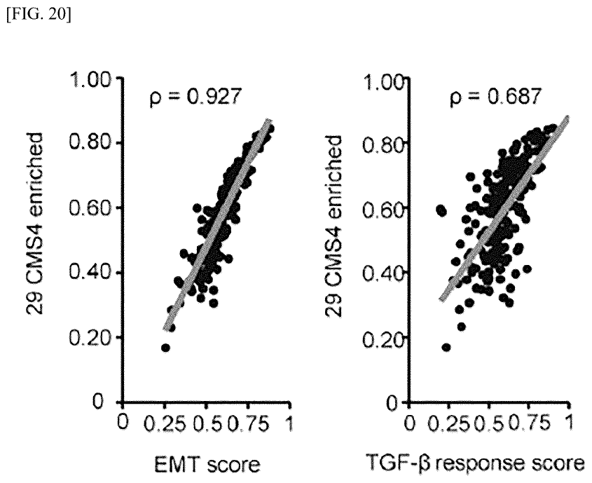

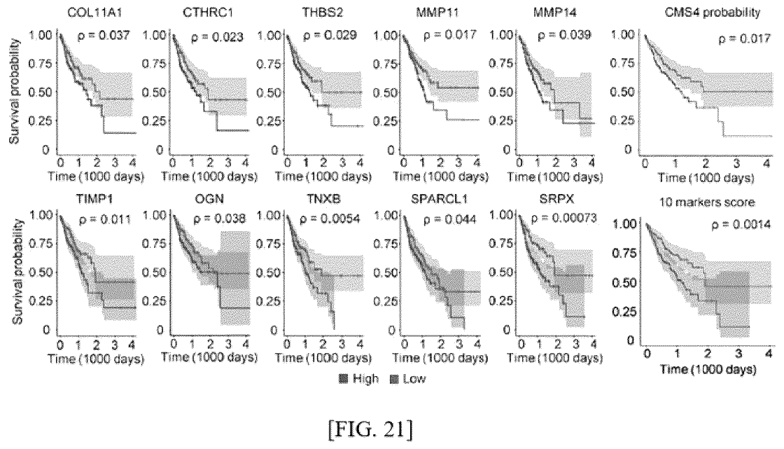

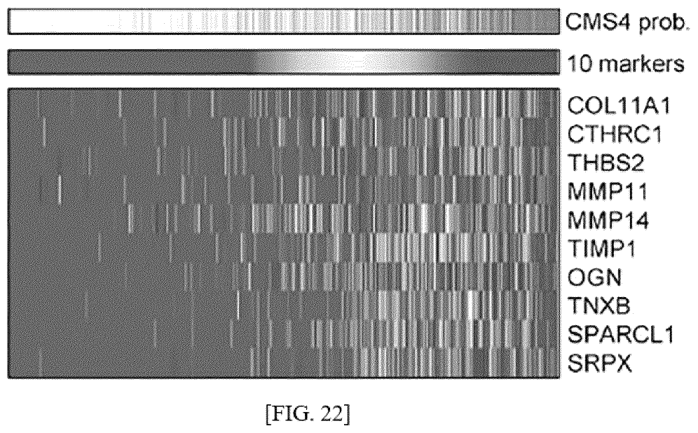

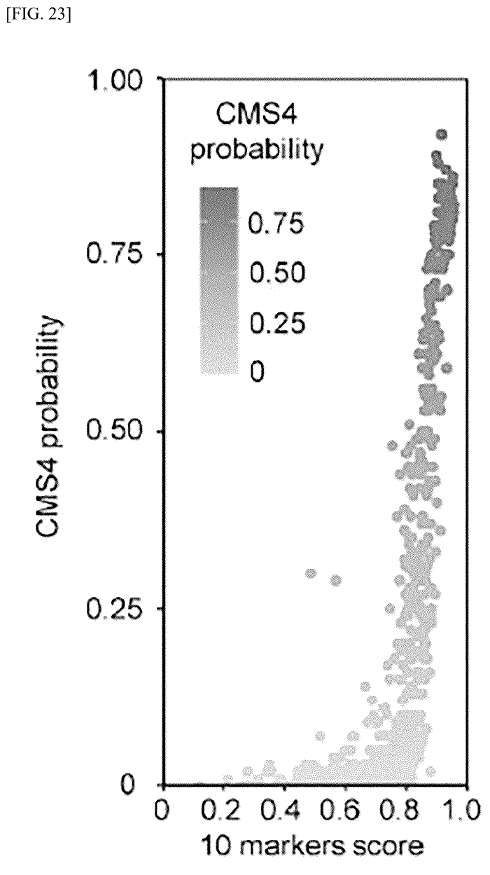

To further characterize the CMS4-specific matrisome features, we performed GSEA with the TCGA dataset used for ssGSEA. Based on the gene set of 38 matrisome markers, GSEA showed significant enrichment of the genes in the CMS4 sample compared with the other subtypes (FIG. 19). Among the 38 matrisome genes, 29 were significantly upregulated in CMS4, comprising 16 TAMs and 13 NAMs. To determine whether these markers were correlated with epithelial-mesenchymal transition (EMT) or TGFβ responses in fibroblasts (i.e., the main characteristics of CMS4), we performed ssGSEA using the markers and gene sets associated with EMT (MSigDB Hallmark M5930) and the TGFβ response in fibroblasts. A scatter plot of the ssGSEA scores of 29 CMS4-enriched markers, EMT score, and TGFβ response score showed stronger correlations between the markers and EMT or the TGFβ response in CMS4, compared with the other subtypes (FIG. 20). Because EMT and the TGFβ response in fibroblasts are associated with lethality, the CMS4-enriched markers may be clinically relevant. To refine the molecular marker based on clinical significance, we performed survival analysis for each marker. Among the 29 CMS4-specific matrisome genes, 10 showed associations with progression-free survival (PFS) (FIG. 21). The 10-gene signature also predicted a poor prognosis, with reduced overall survival and PFS. Similarly, calculating CMS4 probability using the CMSclassifier predicted a poor prognosis, with reduced overall survival and PFS. Furthermore, we found significant correlations between the expression levels of the 10 genes and CMS4 probability (FIG. 22). When the normalized expression score of the 10 genes was <0.7, samples were regarded as the CMS4 subtype. Among the 10 genes, except for SPARCL1 and TIMP1, 8 showed predominant expression in fibroblasts and were highly enriched in CMS4 (FIG. 23). Thus, the 10 clinically significant CMS4-specific matrisome genes may be used to infer the fibroblast population in the TME and to discriminate between CMS4 and other subtypes. Our findings indicate that the activation patterns of the 10 ECM genes are essential in the stroma of CRC, particularly in the CMS4 subtype. These genes may be used to identify the CMS4-specific ECM components that are strongly associated with a poor prognosis.

Although the present disclosure has been described in detail with reference to the specific features, it will be apparent to those skilled in the art that this description is only of a preferred embodiment thereof, and does not limit the scope of the present disclosure. Thus, the substantial scope of the present disclosure will be defined by the appended claims and equivalents thereto.

### INDUSTRIAL APPLICABILITY

The present invention is remarkably effective for accurately diagnosing the most difficult-to-treat and poorly prognostic types of colon cancer, and thus is expected to be widely used in the fields of medicine and health.

1. A method for treating colorectal cancer in a subject, the method comprising;
a step of measuring an expression level of at least one gene, or a protein encoded thereby, selected from the group consisting of COL14A1 (Collagen Type XIV Alpha 1 Chain), DPT (Dermatopontin), MFAP5 (Microfibril Associated Protein 5), MATN2 (Matrilin-2), SRPX (Sushi Repeat Containing Protein X-Linked), MFAP4 (Microfibril Associated Protein 4), MGP (Matrix Gla Protein), TNXB (tenascin XB protein), EDIL3 (EGF Like Repeats And Discoidin Domains 3), LTBP4 (latent transforming growth factor beta binding protein 4), SPARCL1 (SPARC Like 1), OGN (Osteoglycin), HAPLN1 (Hyaluronan And Proteoglycan Link Protein 1), DCN (Decorin), ADAMDEC1 (ADAM like decysin 1), A2M (Alpha-2-Macroglobulin), CTSC (Cathepsin C), CST3 (cystatin c), CXCL12 (C-X-C motif chemokine 12), and S100A4 (S100 Calcium Binding Protein A4), and
a step of treating the subject when the expression level of above is lower than that in a normal control group.

2. The method according to claim 1, further comprising;
a step of measuring an expression level of at least one gene, or a protein encoded thereby, selected from the group consisting of COL12A1 (Collagen type XII α1 chain), COL11A1 (Collagen Type XI Alpha 1 Chain), CTHRC1 (Collagen Triple Helix Repeat Containing 1), FN1 (Fibronectin 1), TNC (Tenascin C), SPARC (Secreted Protein Acidic And Cysteine Rich), THBS2 (Thrombospondin 2), TIMP1 (TIMP Metallopeptidase Inhibitor 1), MMP14 (Matrix Metallopeptidase 14), PLOD2 (Procollagen-Lysine, 2-Oxoglutarate 5-Dioxygenase 2), SERPINH1 (Serpin peptidase inhibitor clade H, member 1), LOXL2 (Lysyl Oxidase Like 2), MMP11 (Matrix Metallopeptidase 11), MMP1 (Matrix Metallopeptidase 1), CTSB (Cathepsin B), MMP3 (Matrix Metallopeptidase 3), LGALS1 (Galectin 1), and SFRP4 (Secreted Frizzled Related Protein 4), and
a step of treating the subject when the expression level of above is higher than that in a normal control group.

3. The method according to claim 1, wherein the colorectal cancer is of the CMS4 (consensus molecular subtype 4) type.

4. The method according to claim 1, wherein the expression level of at least one gene, or a protein encoded thereby, is measured in decellularized tissue.

5. The method according to claim 4, wherein the expression level of at least one gene, or a protein encoded thereby, is measured in decellularized extracellular matrix.

6. A method for screening a therapeutic agent for colorectal cancer, comprising:
(a) a step of treating a biological sample isolated from a target subject with a candidate therapeutic agent for colorectal cancer; and
(b) a step of measuring the expression level of at least one gene, or a protein encoded thereby, selected from the group consisting of COL14A1 (Collagen Type XIV Alpha 1 Chain), DPT (Dermatopontin), MFAP5 (Microfibril Associated Protein 5), MATN2 (Matrilin-2), SRPX (Sushi Repeat Containing Protein X-Linked), MFAP4 (Microfibril Associated Protein 4), MGP (Matrix Gla Protein), TNXB (tenascin XB protein), EDIL3 (EGF Like Repeats And Discoidin Domains 3), LTBP4 (latent transforming growth factor beta binding protein 4), SPARCL1 (SPARC Like 1), OGN (Osteoglycin), HAPLN1 (Hyaluronan And Proteoglycan Link Protein 1), DCN (Decorin), ADAMDEC1 (ADAM like decysin 1), A2M (Alpha-2-Macroglobulin), CTSC (Cathepsin C), CST3 (cystatin c), CXCL12 (C-X-C motif chemokine 12), and S100A4 (S100 Calcium Binding Protein A4).

7. The method according to claim 6, wherein the screening method determines the candidate therapeutic agent as a colorectal cancer treatment if the measured expression level of the protein or gene is increased compared to before the treatment with the candidate agent.

8. The method according to claim 6, wherein the screening method further comprises a step of measuring the expression level of at least one gene, or a protein encoded thereby, selected from the group consisting of COL12A1 (Collagen type XII α1 chain), COL11A1 (Collagen Type XI Alpha 1 Chain), CTHRC1 (Collagen Triple Helix Repeat Containing 1), FN1 (Fibronectin 1), TNC (Tenascin C), SPARC (Secreted Protein Acidic And Cysteine Rich), THBS2 (Thrombospondin 2), TIMP1 (TIMP Metallopeptidase Inhibitor 1), MMP14 (Matrix Metallopeptidase 14), PLOD2 (Procollagen-Lysine, 2-Oxoglutarate 5-Dioxygenase 2), SERPINH1 (Serpin peptidase inhibitor clade H, member 1), LOXL2 (Lysyl Oxidase Like 2), MMP11 (Matrix Metallopeptidase 11), MMP1 (Matrix Metallopeptidase 1), CTSB (Cathepsin B), MMP3 (Matrix Metallopeptidase 3), LGALS1 (Galectin 1), and SFRP4 (Secreted Frizzled Related Protein 4).

9. The method according to claim 8, wherein the screening method determines the candidate therapeutic agent as a colorectal cancer treatment if the measured expression level of the protein or gene is decreased compared to before the treatment with the candidate agent.

10. The method according to claim 6, wherein the colorectal cancer is of the CMS4 (consensus molecular subtype 4) type.

11. The method according to claim 6, wherein the biological sample is a decellularized tissue.

12. The method according to claim 11, wherein the biological sample is a decellularized extracellular matrix.
